(19) United States
(12) Patent Application Publication
(10) Pub. No.: US 2021/0164051 A1
(43) Pub. Date: Jun. 3, 2021

(54) METHODS OF DETECTION AND ANALYSIS OF NUCLEIC ACID IN NEURAL-DERIVED EXOSOMES

(71) Applicant: The Institute for Ethnomedicine dba Brain Chemistry Labs, Jackson, WY (US)

(72) Inventors: Sandra Anne BANACK, Jackson, WY (US); Rachael DUNLOP, Jackson, WY (US); Paul Alan COX, Jackson, WY (US)

(21) Appl. No.: 17/110,206

(22) Filed: Dec. 2, 2020

Related U.S. Application Data
(60) Provisional application No. 63/042,469, filed on Jun. 22, 2020.
(60) Provisional application No. 62/942,682, filed on Dec. 2, 2019.

(51) Int. Cl. C12Q 1/6883

(52) U.S. Cl. CPC C12Q 1/6883; C12Q 2600/178; C12Q 2600/118

(57) ABSTRACT

Presented herein are methods of identifying a subject who has, or is at risk of developing a motor neuron disease comprising determining a presence or amount of one or more neural-derived micro-RNAs (miRNAs). Also presented herein are method of preventing, treating or delaying the onset of a motor neuron disease.

### RELATED APPLICATIONS

This patent application claims priority to U.S. Provisional Patent Application No. 63/042,469 filed Jun. 22, 2020, entitled Methods Of Detection And Analysis Of Nucleic Acid In Neural-Derived Exosomes and naming inventors Sandra Anne Banack, Rachael Dunlop and Paul Alan Cox; and to U.S. Provisional Patent Application No. 62/942,682 filed Dec. 2, 2019, entitled Methods Of Detection And Analysis Of Nucleic Acid In Neurally-Derived Exosomes Fractions naming inventors Sandra Anne Banack, Rachael Dunlop and Paul Alan Cox. The entire contents of the foregoing patent applications are incorporated herein by reference.

### SEQUENCE LISTING

The instant application contains a Sequence Listing which has been submitted electronically in ASCII format and is hereby incorporated by reference in its entirety. Said ASCII copy, created on Jan. 15, 2021, is named 042733-0516320 SL.txt and is 1,524 bytes in size.

### FIELD OF THE INVENTION

Certain embodiments of the invention relate to methods of detecting the presence, absence or amount of one or more miRNAs associated with neural-derived exosomes. Certain embodiments of the invention also relate to specific exosome-derived miRNAs, or subsets thereof, that are predictive and/or diagnostic of a motor neuron disease such as amyotrophic lateral sclerosis (ALS). Certain embodiments of the invention also relate to exosome-derived miRNAs that are useful for monitoring the progression of a motor neuron disease, and/or determining a treatment of a motor neuron disease.

### INTRODUCTION

Exosomes have been shown to comprise nucleic acids, including messenger RNA (mRNA), microRNA (miRNA), and small interfering RNA (siRNA), which can be transferred from one cell to another. Exosomes that are released into the extracellular matrix and taken up by adjacent cells can potentiality transfer information from one cell to another. Such information can be therapeutic or pathogenic.

Presented herein, in certain embodiments, are methods of detection and analysis of miRNAs associated with neural-derived exosomes. In some embodiments, such methods can be used for the diagnosis and early detection of motor neuron diseases, such as ALS.

### SUMMARY

In some aspects, presented herein is method of identifying a subject who has, or is at risk of developing a motor neuron disease comprising: (a) determining a presence or amount of one or more micro-RNAs (miRNAs) in a sample obtained from the subject wherein the one or more miRNA are selected from the group consisting of miR-146a-5p, miR-199a-3p, miR-4454, miR-10b-5p, miR-29b-3p, miR-151a-3p, miR-151a-5p, and miR-199a-5p, and (b) determining if the subject has, or is at risk of developing the motor neuron disease according to the presence or amount of the one or more miRNAs in the sample.

In some aspects, presented herein is a method of preventing or treating a motor neuron disease in a subject who has, or is at risk of developing the motor neuron disease, the method comprising: (a) determining a presence or amount of one or more micro-RNAs (miRNAs) in a sample obtained from the subject wherein the one or more miRNA are selected from the group consisting of miR-146a-5p, miR-199a-3p, miR-4454, miR-10b-5p, miR-29b-3p, miR-151a-3p, miR-151a-5p, and miR-199a-5p; (b) determining if the subject has, or is at risk of developing the motor neuron disease according to the presence or amount of the one or more miRNAs in the sample; and (c) administering a motor neuron disease treatment to the subject when the determining of (b) determines that the subject has, or is at risk of developing the motor neuron disease. In some embodiments, a motor neuron disease treatment comprises administering a therapeutically effective amount of L-serine.

Certain aspects of the technology are described further in the following description, examples, claims and drawings.

### BRIEF DESCRIPTION OF THE DRAWINGS

The drawings illustrate embodiments of the technology and are not limiting. For clarity and ease of illustration, the drawings are not made to scale and, in some instances, various aspects may be shown exaggerated or enlarged to facilitate an understanding of particular embodiments. The file of this patent contains at least one drawing/photograph executed in color. Copies of this patent with color drawing(s)/photograph(s) will be provided by the Office upon request and payment of the necessary fee.

FIG. 1 shows the results of a NanoParticle Tracking Analysis (NTA) comparing Fluorescence NTA (dark red) and Light Scatter NTA (Light blue). Size (nm) is shown on the x-axis and concentration (exp⁶ particles/ml) is shown on the y-axis. The results show that the isolated particles are the correct size for exosomes and demonstrate that Fluorescence NTA is more accurate than Light Scatter NTA for detecting exosomes. The results also show that the neural-enriched exosomes detected (left panel) represent about 1/10 the concentration of total exosomes (right panel).

FIG. 2 shows the results of Western blot array (EXO-CHECK™ Exosome Antibody Array, SBI System Biosciences, Palo Alto, Calif.), used for the detection of exosome and non-exosome markers. The left set of two panels detects the presence or absence of CD63, CD9, CD81, TSG101, CANX and ICAM1 (L1CAM/CD171) (indicated from top to bottom on the left of the blots) in Total-Neural-enriched (Total minus Neural-enriched) exosomes. The right set of two panels detects the presence or absence of L1 Cell Adhesion Molecule (L1CAM/CD171), Neural Adhesion Molecule 1 (NCAM1), Enolase 2 (ENO2), Microtubule Associated Protein Tau (MAPT), Glutamate lonotropic Receptor AMPA Type Subunit 1 (GRIA1) and Proteolipid 1 (PLP1) (from top to bottom on the right set of blots). The results show the absence of cell contamination markers, the present of neural markers in all samples, the presence of exosome internal cellular markers and modest detection of tetraspanins. PC=positive control.

FIG. 3 shows that L1 cell adhesion molecule (L1CAM) expression is enriched in the brain. Expression values are shown in transcripts per million (TPM).

FIG. 4 shows a box-plot representation of variability in gene fold expression [2^{-(ΔΔCt)}] in eight miRNA comparing ALS patient and healthy controls in each of two replicated experiments comprised of separate individual cohorts. All eight miRNAs differed statistically between patients and controls in each of the analyzed cohort experimental groups. A two-tailed Mann-Whitney U Test (non-parametric based on the fact that data distribution plots did not conform to normal distributions) identified statistical differences (p<0.05). ALS.1 and Control.1 (shaded box) represent the first experiment with n=10 samples in each group. ALS.2 and Control.2 (open box) represent an independent replication using a new cohort of patients and controls each with n=10 samples. Center lines show the medians; box limits indicate the 25th and 75th percentiles; whiskers extend 1.5 times the interquartile range from the 25th and 75th percentiles, outliers are represented by circles.

FIG. 5 shows exosome antibody detection of proteins found in neural-enriched exosome (NEE) extracts: Blank is negative control; CD63, CD9, CD81 are tetraspanins (CD63 and CD81 were confirmed independently through ELISAs); TSG101 is a component of the ESCRT-I complex; CANX is a calnexin cell contamination marker; ICAM1 is an intercellular adhesion molecule; PC is a positive control for HRP detection; and neural marker proteins include L1 transmembrane (L1CAM), neural cell adhesion 1 (NCAM1), enolase 2 (ENO2), total tau (MAPT), glutamate receptor 1 (GRIA1) and proteolipid 1 (PLP1).

FIG. 6 shows tissue stained sections of motor neurons. Vervets dosed with rice flour (210 mg/kg/day) for 140 days displayed anterior horn motor neurons with normal cytoarchitecture (A-H). Vervets dosed with the cyanotoxin BMAA (210 mg/kg/day) for 140 days, at an equivalent quantity to that of a 20-year exposure received by a Chamorro of Guam, exhibited shrunken, eosinophilic motor neurons (E) with thinning Nissl substances (F), metabolic stress (G), and abundant skeinlike cytoplasmic vacuoles (E-P). A small proportion of anterior horn motor neurons were observed to have enlarged cell somas with pale cytoplasm, expanded cytoplasmic vacuoles and thinning of Nissl substances (I-L). In addition, Bunina bodies (blue arrow) (M), chromatolysis (K, L, N) glycogen cytoplasmic deposits (white arrows) (K, O) and cell death with neuronophagia (black arrow) (P) were observed. Scale bar: 40 μm.

FIG. 7 shows illustrative images of thioflavin-S/DAPI and Sevier Münger/hematoxylin stained anterior horn motor neurons from vervet primates dosed with rice flour (210 mg/kg/day) or BMAA (210 mg/kg/day) for 140 days. Neurons from rice flour-dosed cohorts were negative for thioflavin-S fluorescence and Sevier Munger staining (A, F). In BMAA-dosed cohorts thioflavin-S and Sevier Münger staining displayed mild to moderate insoluble and argyrophilic intraneuronal inclusions in motor neurons ranged from diffused (B, G), filamentous (C, H), to dense (D, I) and granular types (E, J). Scale bar: 25 μm (A) and 40 μm (B-J).

FIG. 8 shows tissue staining of motor neurons demonstrating proteinopathies including: representative microscopic images of spinal cord anterior horn motor neurons from control vervets immunoprobed with anti-TDP-43 (A); classic granular phosphorylated TDP-43+ intracytoplasmic inclusions observed in anterior horn motor neurons of vervets dosed with the BMAA neurotoxin (210 mg/kg/day) for 140 days (B); dense, rounded, and spicule-like TDP-43+ cytoplasmic inclusion bodies (C, D); motor neurons of rice flour controls displaying predominant nuclear staining of FUS protein (E); increased cytoplasmic translocation of FUS+ nuclear protein in BMAA-dosed vervets (blue arrow)(F); representative high-powered images of anterior horn motor neurons with pathological cytoplasmic FUS+ inclusions (G, H); anterior horn motor neurons from rice flour control vervets probed with anti-UBIQ (I); ALS/MND-type UBIQ+ inclusions in neuronal cell bodies and nuclei (black arrow), neurites (white arrow), and glia in BMAA-dosed vervets (J-L); and high magnification of a dense UBIQ+ deposit shown in the small dotted square #1 in (J) as shown in (K) and high magnification of a UBIQ+ microglia from large dotted square #2 in (J) as shown in (L). Scale bars: 250 μm (A, B, E, F), 50 μm (C, D, G, H, K, L), and 300 μm (I, J).

FIG. 9 shows tissue staining of reactive astrogliosis and quantitation thereof including: GFAP+ astroglial cell bodies and processes in rice flour-dosed controls (210 mg/kg/day) that are compact and proximate to intact anterior horn motor neurons (A); high-powered digital pathology scans of astroglia labeled in blue dotted squares (B, C); severe reactive GFAP+ astroglia observed adjacent to shrunken and vacuolated anterior horn motor neurons in vervets dosed with the BMAA toxin (210 mg/kg/day) for 140 days (D-F); comparative digital pathology scans of GFAP+ astroglia from a patient with sporadic ALS (sALS)(G-I); mild effects of BMAA dosing on the cervical spinal cord (NS, p=0.427; n=8)(J); and BMAA-induced increases of the median numbers of GFAP+ astroglia in the anterior horn of the lumbar spinal cord by 44% (**p=0.008; n=8) (K). Coadministration of L-serine reduced the numbers of the GFAP+ astroglia by 21%. Automated image analysis of the anterior horn show that BMAA effects were reduced 20% by coadministration of L-serine (**p=0.0078; n=8) (L). Scale bars: 100μm (A, D, G) and 25 μm (B, C, E, F, H, I).

FIG. 10 shows tissue staining demonstrating microglial activation. Vervets dosed with rice flour (210 mg/kg/day) for 140 days showed a normal distribution of quiescent microglia in anterior and posterior horns and both ascending and descending white matter tracts of the cervical spinal cord(A-C). Vervets dosed with the cyanobacterial toxin BMAA (210 mg/kg/day) displayed an increased density of activated IbA1+ microglia in ascending motor neuron pathways (blue arrow)(D). Large focal and bilateral nodules of IbA1+ microglia were observed in the lateral corticospinal tracts (dotted squares) (E). Microglial nodules (black arrow) varied in sized and distribution adjacent to a vacuolated neuropil. Also shown is a microglial nodule visualized with H&E staining (F)(Insert 1); corticospinal tracts, lateral (blue arrow) and anterior (white arrow), that were positive for CD68, a marker for proinflammatory microglial activation(G); large focal nodules of CD68+ microglia (dotted square) (H); predominate focal (black arrow) and foamy phagocytic (Insert 2) microglial nodules that were present in BMAA toxin-dosed cohorts (I); a patient with sporadic ALS (sALS) displaying predominately diffused CD68+ microglial immunostaining evident of advanced disease stage (J-L); and an Iba1+ microglial nodule from a sALS patient (Insert 3). Scale bars: 1200 μm (left panel A-G), 1500 μtm (left panel J), 500 μm (middle panel B-K), and 250 μm (right panel C-L).

FIG. 11 data demonstrating L-Serine neuroprotection in ALS including topographical maps illustrating manual regional analysis of microglial activation in the cervical spinal cord for rice flour, BMAA, or BMAA+L-serine-dosed vervets (A-C). Each vervet is identified by a unique colored dot representing a large area of Iba1+ microglial activation or nodule. BMAA-dosed vervet cohorts had increased bilateral microglial activation in the lateral corticospinal tracts. Adjacent anatomical regions analyzed were unaffected. To determine the therapeutic effects of L-serine, automated image analyses were performed on lateral corticospinal tracts of each dosing cohort. Chronic dietary dosing with BMAA (210 mg/kg/day) for 140 days increased the median density (pixel counts) by 1.7-fold (*p=0.048; n=8) (D) and the total area of the lateral corticospinal tracts covered by IbA1+ microglia by 1.6-fold (*p=0.011; n=8) (E). BMAA dosing did not have a significant effect on the size of Iba1+ microglia in the lateral corticospinal tracts (NS, p=0.260; n=8) (F). The distribution of CD68+ microglial nodule number and size (***p<0.0001; n=8) was increased in the cervical and lumbar (spinal cord) segments of toxin-exposed primates (G). Coadministration of L-serine also reduced the effects of BMAA on CD68+ microglial nodule numbers, size, and expression intensity (**p=0.01; n=8) (H).

FIG. 12 shows digitized pathology scans of a Luxol fast blue (LFB) counterstained with hematoxylin and eosin (H&E) spinal cord segment shown in low (3.3×), medium (11×), and high power (40×) from a vervet dosed with rice flour for 140 days (A, D, G) and vervet cohorts dosed with the cyanotoxin, BMAA (210 mg/kg/day), for 140 days displaying pallor of staining of myelinated axon fibers in the lateral corticospinal tracts (dotted square) (B, E, H). Loss of myelinated axon fiber staining was observed in 7 of 16 (44%) (NS, p=0.0956; n=8) of vervets exposed to BMAA. Representative images of LFB/H&E-stained spinal cord segment from an individual with sALS are shown for comparison (C, F, I). Scale bar: 1300 μm (A, B), 1900 μm (C), and 400 μm (D-F).

FIG. 13 shows representative digital pathology scans of an NFT and a neuritic plaque observed in the cerebral cortex of vervets fed with BMAA (210 mg/kg/day) for 140 days (A) and the median cortical tau AT8+ NFT density as calculated by averaging automated density counts across the following 7 regions: cingulate, entorhinal, frontal, insular, motor, occipital, and temporal cortex (B). BMAA dosing increased the median density of AT8+ NFTs 3.1-fold (***p<0.0001; n=8). The production of BMAA-induced tauopathy was partially block by coadministration of L-serine. Cortical AT8+ NFT deposition in BMAA-dosed vervets was positively correlated with detectable levels of total BMAA in spinal cord tissues (r=0.6804; ***p=0.0004; n=23) (C); the total area of IbA1+ microglia in the lateral corticospinal tract (r=0.6399; ***p=0.0003; n=24)(D); the number of TDP-43-positive cytoplasmic inclusions in anterior horn motor neurons (r=0.4905; *p=0.01; n=24)(E); and the number of GFAP+ astroglia adjacent to motor neurons (r=0.6488; ***p=0.0004; n=24)(F). Coadministration of L-serine with BMAA reduced the observed corticospinal neuropathology in the cerebral cortex and spinal cord of BMAA-dosed vervets. The solid black lines on scatter plots represent the best-fit line at 95% confidence interval. The red dotted lines indicate error. Scale bar: 25 μm.

### DETAILED DESCRIPTION

As described herein, the miRNA content of neural-derived exosomes (e.g., neural-enriched exosome fractions) has diagnostic and therapeutic utility. The abundance of, for example, tetraspanins and cell adhesion molecules (CAMs) expressed on or in exosomes derived from neural cells or neural tissues makes it possible to detect, enrich, prepare, and/or isolate neural-derived exosomes. Such neural-derived exosomes may be employed to detect and/or quantify the amount of certain exosome-derived miRNAs. The presence or amount of certain exosome-derived miRNAs, or sets of such miRNAs, provides insight as to whether a subject has, or is at risk of developing certain motor neuron diseases. For example, the presence or amount of certain exosome-derived miRNAs can also be used to provide early diagnosis of a motor neuron disease and/or to identify subjects who are at risk of developing a motor neuron disease. Accordingly, in some embodiments, methods are provided herein for treatment of asymptomatic subjects who are at risk of developing a motor neuron disease. Such methods can inhibit the progression of a motor neuron disease, or delay the onset of a motor neuron disease.

Amyotrophic Lateral Sclerosis (ALS), a non-limiting example of a motor neuron disease, is the most common form of a motor neuron disease (MND). ALS/MND, or Lou Gehrig's disease, is a progressive motor neuron disease characterized by death of both upper and lower motor neurons and subsequent muscle atrophy. There are a variety of genetic and environmental risk factors that may lead to ALS which is believed to result from gene/environment interactions and this serious illness likely represents a syndrome rather than a single disease. The onset of ALS symptoms often presents a crisis for patients and their families, with time from diagnosis to death having a mean of 2.5 to 3 years, although some patients persist much longer. Loss of functionality due to progressive motor neuron loss leads to ataxia, aphasia, muscle spasticity, muscle fasciculations, and progressive paralysis. Some patients also suffer cognitive deficits.

One of the problems in current ALS therapy is slowness of diagnosis. In its initial presentation, ALS is often misdiagnosed. Few general practitioners feel comfortable in making a possible or probable diagnosis of ALS and so refer patients to neurologists, who in turn typically use progressive deterioration of upper and lower motor neuron function and increasing muscle atrophy as measured through time as indicative of ALS. As a result, diagnosis of ALS typically takes months, and sometimes a year or more, to be made. This precious time lost due to the inability to diagnose presents a significant burden on patients and their families, as well as their physicians, because of the inability to prescribe medication or even to plan for treatment and patient care. At any one time in the United States, there are 25,000 to 30,000 living patients diagnosed with ALS, but because they perish so rapidly, they have little political clout. As a result, it is left to academic departments and small not-for-profit organizations such as the Brain Chemistry Labs to perform the necessary research.

Neural-derived exosome fractions, and/or fractions comprising portions or components of exosomes of neural cell or neural tissue origin (individually and collectively referred to interchangeably as “a neural-derived exosome fraction” or “neural-enriched exosome fractions”), may be detected, isolated, enriched, or prepared from a sample, for example, a sample obtained from a subject.

As the skilled artisan will appreciate, a variety of enrichment methods, process, and reagents may be employed in order to prepare neural-derived exosomes or neural-enriched exosome fractions from samples. Neural-derived exosomes or neural-enriched exosome fractions can be isolated and/or prepared from a sample using a suitable method.

In some embodiments, such neural-enriched exosome fractions comprise exosomes that are neural-derived exosomes, non-limiting examples of which include neuron-derived exosomes, astrocyte-derived exosomes, oliogodendrocyte-derived exosomes, microglia-derived exosomes, combinations thereof and the like.

In some embodiments, methods for preparing neural-enriched exosome fractions may comprise enriching for exosomes of neural cell or neural tissue origin based on differences in the biochemical and/or physiochemical properties of exosomes. For example, neural-derived exosomes may be prepared from samples by enriching for exosomes based on antigen and/or centrifugal differences. In some of the embodiments based on antigen differences, antibody-conjugated magnetic or paramagnetic beads in magnetic field gradients or fluorescently labeled antibodies with flow cytometry may be used to prepare neural-enriched exosome fractions.

In some of the embodiments flow cytometry may be employed in methods for preparing neural-enriched exosome fractions. In some of the embodiments, dye uptake/exclusion measured by flow cytometry or another sorting technology may be employed to prepare neural-enriched exosome fractions. In some embodiments, cell culture with cytokines may be employed to prepare neural-enriched exosome fractions.

Neural-enriched exosome fractions may also be prepared from samples based on other biochemical properties such as pH or motility.

In some embodiments, combinations of one or more of the methods described herein and throughout may be employed in methods for preparing neural-enriched exosome fractions from samples.

A “sample” or “samples”, as used interchangeable herein, is often obtained from a suitable subject. A sample can be isolated or obtained directly from a subject or part thereof. In some embodiments, a sample obtained from a subject is a sample derived from the subject. Accordingly, in certain embodiments, a sample obtained from a subject is a sample obtained directly from the subject. In certain embodiments, a sample obtained from a subject is obtained from a third party, for example a third party who obtained or extracted the sample from the subject. In some embodiments, a sample is obtained indirectly from an individual or a medical professional. A sample can be any specimen that is isolated or obtained from a subject or part thereof. A sample can be any tissue or fluid that is isolated or obtained from one or more subjects. Non-limiting examples of samples include fluids or tissues obtained or derived from a subject, including, without limitation, blood or a blood product (e.g., serum, plasma, platelets, buffy coats, lymphatic fluid or the like), umbilical cord blood, chorionic villi, amniotic fluid, cerebrospinal fluid (CSF), spinal fluid, lavage fluid (e.g., lung, gastric, peritoneal, ductal, ear, arthroscopic), a biopsy sample, celocentesis sample, cells (blood cells, lymphocytes, placental cells, stem cells, bone marrow derived cells, embryo or fetal cells, neurons) or parts thereof (e.g., mitochondrial, nucleus, extracts, lysates, or the like), urine, feces, sputum, saliva, nasal mucous, prostate fluid, lavage, semen, lymphatic fluid, bile, tears, sweat, breast milk, breast fluid, the like or combinations thereof. In some embodiments, a sample comprises or is suspected of comprising exosomes (e.g., neural-derived exosomes), or portions or components thereof.

Non-limiting examples of subjects include mammals, humans, non-human primates (e.g., apes, gibbons, chimpanzees, orangutans, monkeys, macaques, and the like), domestic animals (e.g., dogs and cats), farm animals (e.g., horses, cows, goats, sheep, and pigs) and experimental animals (e.g., mouse, rat, rabbit, and guinea pig). In some embodiments a subject is a mammal. A mammal can be any age or at any stage of development (e.g., an adult (e.g., 18, 19, 20 or 21 years and older), a senior adult (e.g., over the age of 55, over the age of 60, or over the age of 65 years), a teen (e.g., age 12 to 19 yrs.), child (e.g., age 1 to 12 yrs.), infant (e.g., from birth to 1 yr.), or a mammal in utero). A mammal can be male or female. In some embodiments, a subject is a human.

In some embodiments, a subject has, is suspected of having, or is at risk of developing, a motor neuron disease. In some embodiments the motor neuron disease is ALS. In some embodiments, a subject who has a motor neuron disease is a subject diagnosed as having a motor neuron disease by a medical professional (e.g., a medical doctor) based on, for example the presence of one or more diagnostic symptoms and/or the results of one or more standardized diagnostic test results. A subject suspected of having a motor neuron disease is a subject not yet diagnosed as having a motor neuron disease by a medical professional. In some embodiments, a subject suspected of having a motor neuron disease may display one or more symptoms of a motor neuron disease, which symptoms are not conclusive evidence that the subject has a motor neuron disease. In some embodiments, a subject who is suspected of a having a motor neuron disease may have one or more symptoms of a motor neuron disease, but is not diagnosed as having any one particular motor neuron disease because there is not enough data to indicate conclusively that the subject has a particular motor neuron disease. In some embodiment, a subject who is suspected of having a motor neuron disease is a subject suspected of having ALS, but is not diagnosed as having ALS by a medical professional.

In some embodiments, a subject is determined to be a subject at risk of developing a motor neuron disease by conducting a method described herein. In some embodiments, a subject at risk of developing a motor neuron disease is a subject who is asymptomatic for a motor neuron disease. In some embodiments, a subject at risk of developing a motor neuron disease is a subject having one or more symptoms of a motor neuron disease, which symptoms may be mild or transient in nature. In some embodiments, a subject at risk of developing a motor neuron disease is a subject who is not yet diagnosed as having a motor neuron disease. In some embodiments, a subject at risk of developing a motor neuron disease is a subject suspected of having a motor neuron disease.

In some embodiments a method described herein identifies a subject who has or is at risk of developing a Motor neuron disease (MND). In some embodiments a method described herein is a method of treating a subject has or is at risk of developing a Motor neuron disease (MND). MNDs are often described as group of progressive neurological disorders that destroy motor neurons, the cells that control skeletal muscle activity such as walking, breathing, speaking, and swallowing. Non-limiting examples of a motor neuron disease include Amyotrophic Lateral Sclerosis (ALS), progressive bulbar palsy (PBP), progressive muscular atrophy (PMA), primary lateral sclerosis (PLS), spinal muscular atrophy (Type I, II and II), spinal muscular atrophy with respiratory distress type 1, Kennedy's disease, and post-polio syndrome. In some embodiments, a motor neuron disease is Amyotrophic Lateral Sclerosis (ALS). In certain embodiments, a motor neuron disease is not Alzheimer's disease (AD). In certain embodiments, a motor neuron disease is not Parkinson's disease (PD). In certain embodiments, a motor neuron disease is not a disease selected from Huntington's disease (HD), multiple sclerosis, Pick's disease, spinocerebellar atrophy, or Machado-Joseph's disease. In certain embodiments, a motor neuron disease is not Dentatorubropallidoluysian atrophy (DRPLA). In certain embodiments, a motor neuron disease is not Creutzfeldt-Jakob's disease or Lewy body disease.

In some embodiments a method described herein identifies a subject who has or is at risk of developing a neurological disease selected from Alzheimer's disease (AD), Parkinson's disease (PD), Huntington's disease (HD), multiple sclerosis, Pick's disease, spinocerebellar atrophy, Machado-Joseph's disease, Dentatorubropallidoluysian atrophy (DRPLA), Creutzfeldt-Jakob's disease, Lewy body disease, and the like. In some embodiments a method described is a method of treating a subject who has or is at risk of developing a neurological disease selected from Parkinson's disease (PD), Huntington's disease (HD), multiple sclerosis, Pick's disease, spinocerebellar atrophy, Machado-Joseph's disease, Dentatorubropallidoluysian atrophy (DRPLA), Creutzfeldt-Jakob's disease, Lewy body disease, and the like.

In certain embodiments, the presence or absence of a motor neuron disease in a subject is determined by a method herein. In some embodiments, wherein a subject is determined to have a motor neuron disease that is diagnosed by a method herein, the method further comprises administering a suitable treatment to the subject, wherein the motor neuron disease, or one or more symptoms thereof are therapeutically treated. In certain embodiments, a method herein identifies a subject who is at risk of developing a motor neuron disease. In some embodiments, wherein a subject is identified as at risk of developing a motor neuron disease by a method herein, the method further comprises administering a suitable treatment to the subject, wherein the motor neuron disease, or one or more symptoms thereof are therapeutically treated. In some embodiments, a method of treating a motor neuron disease is a method of inhibiting or delaying the onset or progression of the motor neuron disease, for example in a subject at risk of developing a motor neuron disease. In some embodiments, a method comprises treating a motor neuron disease or one or more symptoms thereof. In some embodiments, a method of treating a motor neuron disease is a method of inhibiting or delaying the onset or progression of one or more symptoms of a motor neuron disease, for example in a subject at risk of developing a motor neuron disease.

Non-limiting examples of a symptom of a motor neuron disease include a motor deficiency; fatigue (e.g., excessive fatigue); passivity; lethargy; inertia; tremors; ataxia; speaking difficulty (e.g., slurred, thick or irregular speech); muscle cramps (e.g., excessive muscle cramping, not necessarily induced by excessive use or excessive exercise), twitching, atrophy or weakness; shortness of breath; breathing difficulty; writing difficulty; unusual or frequent stiffness or rigidity; loss of fine or gross motor control; slowing of movement; impaired balance; body instability; posture or gait abnormality (e.g., shuffling walk, unsteady or irregular gait); reduced coordination; motor dysfunction; jerky or involuntary body movement; slowed saccadic eye movement; seizures; difficulty chewing, eating, or swallowing; loss of balance; opthalmoparesis or impaired eye movement; impaired eyelid function; involuntary facial muscle contracture; neck dystonia or backward tilt of the head with stiffening of neck muscles; urinary/bowel incontinence; parkinsonism; the like and combinations thereof.

In some embodiments, a method comprises preventing or treating a motor neuron disease, inhibiting or delaying the onset of, or progression of a motor neuron disease, or inhibiting, mitigating, reducing or delaying the onset of one or more symptoms of a motor neuron disease, where the method comprises administering a therapeutically effective amount of a motor neuron disease drug, non-limiting examples of which include L-serine, ralitoline, phenytoin, lamotrigine, carbamazepine, lidocaine, tetrodotoxin, nitroindazole, a sulforaphane or sulforaphane analogue, gabapentin, pregabalin, Mirogabalin, gabapentin enacarbil, phenibut, imagabalin, atagabalin, 4-methylpregabalin, PD-217,014, Riluzole, Edaravone, tetrabenazine, haloperidol, risperidone, quetiapine, amantadine, levetiracetam, clonazepam, citalopram, escitalopram, fluoxetine, sertraline, quetiapine, risperidone, olanzapine, valproate, carbamazepine, lamotrigine, a vaccine (e.g., an immunogenic amount of an amyloid peptide, or a fragment or variant thereof, with or without an adjuvant), a cholinesterase inhibitor (e.g., donepezil, galantamine or rivastigmine), memantine, an antidepressant, an N-methyl D-aspartate (NMDA) antagonist, an omega-3 fatty acid, curcumin, or a curcumin derivative, vitamin E, a sleep aid (e.g., zolpidem, eszopiclone or zaleplon), an anti-anxiety drug (e.g., lorazepam and clonazepam), an anti-convulsant (e.g., sodium valproate, carbamazepine, or oxcarbazepine), an anti-psychotic (e.g., risperidone, quetiapine or olanzapine), carbidopa-levodopa, amantadine, a dopamine agonists (e.g., pramipexole, ropinirole, rotigotine or Apomorphine), a MAO B inhibitor (e.g., selegiline, rasagiline or safinamide), a Catechol O-methyltransferase (COMT) inhibitors (e.g., entacapone or tolcapone), an anticholinerigic (e.g., benztropine or trihexyphenidyl), the like and combinations thereof. In some embodiments, a motor neuron disease is treated by a method comprising administering a therapeutically effective amount of one or more of L-serine, Riluzole, Edaravone, Nusinersen, Onasemnogeme abeparovec-xioi (ZOLGENSMA™), Radicava, Rilutek, Tiglutik, Nuedexta, muscle relaxers (e.g., baclofen, tizanidine, benzodiazepines) or botulinum toxin. In some embodiments, a motor neuron disease is treated by a method comprising administering a therapeutically effective amount of L-serine.

In some embodiments, where the motor neuron disease is ALS, a treatment comprises administered a therapeutically effective amount of ralitoline, phenytoin, lamotrigine, carbamazepine, lidocaine, tetrodotoxin, Riluzole, Edaravone, Gabapentin, pregabalin, Mirogabalin, gabapentin enacarbil, phenibut, imagabalin, atagabalin, 4-methylpregabalin, PD-217,014, Trihexyphenidyl, amitriptyline, baclofen, diazepam, L-serine, CK-2127107 (reldesemtiv), Nusinersen, Onasemnogeme abeparovec-xioi (ZOLGENSMA™), Radicava, Rilutek, Tiglutik, Nuedexta, the like or combinations thereof. In some embodiments, where the motor neuron disease is ALS, a treatment comprises administered a therapeutically effective amount of L-serine.

In some embodiments, where a neurological disease is HD, a treatment comprises administered a therapeutically effective amount of Tetrabenazine, haloperidol, risperidone, quetiapine, amantadine, levetiracetam, clonazepam, citalopram escitalopram, fluoxetine, sertraline, quetiapine, risperidone, olanzapine, valproate, carbamazepine, or lamotrigine.

In some embodiments, where a neurological disease is PD, a treatment comprises administered a therapeutically effective amount of Carbidopa-levodopa, amantadine, a dopamine agonists (e.g., pramipexole, ropinirole, rotigotine or Apomorphine), a MAO B inhibitor (e.g., selegiline, rasagiline and safinamide), a Catechol O-methyltransferase (COMT) inhibitor (e.g., Entacapone or Tolcapone), an anticholinerigic (e.g., benztropine or trihexyphenidyl), the like or combinations thereof.

L-Serine

In some embodiments, a subject is administered a therapeutically effective amount of L-serine, a salt, metabolic precursor, derivative or conjugate thereof. In some embodiments, a subject is administered a therapeutically effective amount of free L-serine, or a salt thereof. A therapeutically effective amount of L-serine or free L-serine may be administered as a pharmaceutical composition comprising one or more pharmaceutical excipients, additives, carriers and/or diluents. In some embodiments, a method herein comprises administered a therapeutically effective amount of a composition comprising, consisting of, or consisting essentially of L-serine, a salt, metabolic precursor, derivative or conjugate thereof to a subject. In some embodiments, a method herein comprises administered a therapeutically effective amount of a composition comprising, consisting of, or consisting essentially of free L-serine, or a salt, derivative or conjugate thereof to a subject. In some embodiments, a method herein comprises administered a therapeutically effective amount of a composition comprising, consisting of, or consisting essentially of a polymer of L-serine, or a salt, derivative or conjugate thereof to a subject. In some embodiments, a composition consisting essentially of L-serine, free L-serine, or a salt, a precursor, a derivative or a conjugate thereof excludes proteins or protein fractions comprising less than 100%, 99%, 98%, less than 95%, less than 90%, less than 80%, less than 70%, less than 60%, or less than 50% L-serine (wt/wt). In some embodiments, a composition consisting essentially of L-serine, free L-serine, or a salt, a precursor, a derivative or a conjugate thereof excludes proteins or protein fractions comprising greater than 5%, greater than 10%, greater than 20%, greater than 30%, greater than 40%, greater than 50% or greater than 60% protein (wt/wt). In some embodiments, a composition consisting essentially of L-serine comprises free L-serine, or a polymer of L-serine having an amino acid content of L-serine of at least 100%, 99%, 98%, 95%, 90%, 85% or at least 80%. In some embodiments, a composition consisting essentially of L-serine excludes creatine, creatine pyruvate, guanidino-acetic acid (GA), glycocyamine, N-amidinoglycine, and salts or esters thereof. In some embodiments, a composition consisting essentially of L-serine is a composition comprising free L-serine at a purity of at least 85%, at least 90%, at least 95%, at least 98%, at least 99% or 100%. In certain embodiments, a composition consisting essentially of L-serine, free L-serine, or a salt, a precursor, derivative or conjugate of L-serine, is a composition that also comprises zinc.

Free L-serine refers to L-serine in the form of a single amino acid monomer, or a salt thereof. In some embodiments, a composition comprises free L-serine at a purity of at least 85%, at least 90%, at least 95%, at least 98%, at least 99% or 100%. In certain embodiments, free L-serine is not covalently bonded to any other amino acid.

In some embodiments, a composition comprising L-serine may exclude other active ingredients. In some embodiments, a composition may exclude proteins containing L-serine. In some embodiments, a composition may exclude proteins having a molecular weight greater than 10 kDa, greater than 20 kDa, greater than 30 kDa or greater than 50 kDa. In some embodiments, a composition may exclude proteins containing less than 99%, 98%, 95%, 92%, 90%, 80%, 70%, 60%, or less than 50% L-serine. In some embodiments, a composition may exclude creatine, or any energy metabolism precursor of creatine, such as guanidino-acetic acid (GA), equivalents thereof, and mixtures thereof.

In certain embodiments, a composition comprises L-serine, non-limiting examples of which include free L-serine, and polymers or polypeptides comprising at least a 50%, 55%, 60%, 65%, 70%, 75%, 80%, 85%, 86%, 87%, 88%, 89%, 90%, 91%, 92%, 93%, 94%, 95%, 96%, 97%, 98%, 99%, or 100% L-serine by weight or amino acid content. In some embodiments, a polymer of L-serine or a polypeptide comprising L-serine includes between 2 and 50000, between 2 and 500, between 2 and 100, between 2 and 50, between 2 and 20, between 2 and 15, between 2 and 10, between 2 and 9, between 2 and 8, between 2 and 7, between 2 and 6, between 2 and 5, or between 2 and 4 L-serine amino acids linked by covalent bonds. In certain embodiments, a composition comprises L-serine, non-limiting examples of which include a polymer or polypeptide comprising from 20% to 100%, from 30% to 100%, from 35% to 100%, from 40% to 100%, from 45% to 100%, from 50% to 100%, from 55% to 100%, from 60% to 100%, from 65% to 100%, from 70% to 100%, from 75% to 100%, from 80% to 100%, from 85% to 100%, from 90% to 100%, from 95% to 100%, from 96% to 100%, from 97% to 100%, from 98% to 100%, or from 99% to 100% content of L-serine (wt/wt) or amino acid content (i.e., L-serine monomers/total amino acid monomers).

Non-limiting examples of a salt of L-serine include a sodium salt, potassium salt, calcium salt, magnesium salt, zinc salt, ammonium salt; inorganic salts such as, hydrogen chloride, sodium chloride, potassium chloride, calcium chloride, sodium phosphate, potassium phosphate, and sodium hydrogen carbonate; organic salts such as, sodium citrate, citrate, acetate, and the like. In certain embodiments, a composition comprises L-serine as an alkylated L-serine, such as L-serine with an alkyl group, or e.g., an alkyl comprising 1-20 carbon atoms. In certain embodiments, a derivative of L-serine includes an L-serine ester, an L-serine di-ester, a phosphate ester of L-serine, or a sulfate or sulfonate ester of L-serine. Non-limiting examples of a conjugate of L-serine includes a pegylated L-serine (e.g., an L-serine comprising one or more polyethylene glycol (PEG) moieties), and a lipidated L-serine. Non-limiting example of a precursor of L-serine include L-phosphoserine.

Non-limiting examples of a precursor of L-serine include a pro-form of L-serine that is broken down into L-serine monomers by the digestive system of a subject. In some embodiments, L-serine or a conjugate thereof consists of a slow-release version. In some embodiments a derivative of L-serine is conjugated to a different molecule forming a prodrug from which L-serine is released after crossing the blood/brain barrier.

In some embodiments, a composition consisting essentially of L-serine may comprise some amount of D-serine. For example, a composition consisting essentially of L-serine may include a small amount of D-serine, for example, less than 30%, less than 25%, less than 20%, less than 15%, less than 10%, less than 9%, less than 8%, less than 7%, less than 6%, less than 5%, less than 4%, less than 3%, less than 2%, less than 1%, less than 0.9%, less than 0.8%, less than 0.7%, less than 0.6%, less than 0.5%, less than 0.4%, less than 0.3%, less than 0.2%, or less than 0.1% D-serine by weight (e.g., wt/wt) or amino acid content (e.g., L-serine/total amino acid content). For example, a composition may include from 0.001% to 30%, from 0.005% to 30%, from 0.1% to 30%, from 1% to 30%, from 2% to 30%, from 3% to 30%, from 4% to 30%, from 5% to 30%, from 6% to 30%, from 7% to 30%, from 8% to 30%, from 9% to 30%, from 10% to 30%, from 0.001% to 20%, from 0.005% to 0%, from 0.1% to 20%, from 1% to 20%, from 2% to 20%, from 3% to 20%, from 4% to 20%, from 5% to 20%, from 6% to 20%, from 7% to 20%, from 8% to 20%, from 9% to 20%, or from 10% to 20% D-serine. In some embodiments, a composition comprising or consisting essentially of L-serine, does not comprise a substantial amount of D-serine. In some embodiments, a composition comprising or consisting essentially of L-serine, does not contain D-serine.

Neural-Derived Exosomes

In some embodiments, neural-derived exosomes comprise neural-derived miRNAs. In some embodiments, neural-derived exosomes comprise or express a neuro-specific polypeptide, non-limiting examples of which include tetraspanins, for example, CD9, CD63, and CD81; and cell adhesion molecules, for example, L1CAM/CD171, a cadherin, a nectin, a sidekick cell adhesion molecule, an integrin, a neuroligin, a neuroexin, an ephrin, Syg-1, Syg-2, NCAM/CD56, and/or combinations thereof. In some embodiments, neural-derived exosomes comprise or express one or more tetraspanins. In some embodiments, neural-derived exosomes comprise or express one or more of CD9, CD63, and CD81. In some embodiments, a neural-derived exosome is CD9+, CD63+ and/or CD81+. In some embodiments, a neural-derived exosome is L1CAM/CD171+. In some embodiments, a neural-derived exosome is an exosome comprising or expressing a tetraspanin. In some embodiments, a neural-derived exosome is an exosome comprising or expressing CD9, CD63 and/or CD81. In some embodiments, a neural-derived exosome is an exosome comprising or expressing L1CAM/CD171.

In some embodiments, neural-enriched exosome fractions and/or neural-derived exosomes are detected, enriched, isolated or purified from a sample, or a neural-enriched exosome fraction, using a suitable method. In some embodiments, neural-enriched exosome fractions are prepared using one or more antibodies, or similar binding agents, that specifically bind to a neuro-specific polypeptide, using a suitable method. In some embodiments, neural-enriched exosome fractions are prepared by a process comprising contacting a sample comprising neural-enriched exosome fractions with one or more antibodies that specifically bind to a protein or marker that is expressed or found on the surface of a neural exosome, thereby forming a plurality of exosome/binding agent complexes. In some embodiments a neural-enriched exosome fraction comprising a plurality of exosome/antibody complexes is prepared by a process comprising immunoprecipitation.

In some embodiments, neural-derived exosomes or neural-enriched exosome fractions are detected, isolated or prepared by a process comprising contacting a sample comprising exosomes with an antibody, or similar binding agent, that specifically binds to L1CAM/CD171. In some embodiments the process comprises contacting a sample comprising exosomes with an antibody that specifically binds to L1CAM/CD171. In certain embodiments a sample is contacted with an anti-L1CAM/CD171 antibody, thereby forming a complex comprising the antibody, L1CAM/CD171 and a neural-enriched exosome, followed by detection, enrichment or isolation of the complex using a suitable method. In some embodiments a plurality of anti-L1CAM/CD171/exosome complexes are isolated by a process comprising immunoprecipitation.

In certain embodiments, a binding agent comprises or consists of one or more polypeptides or one or more proteins that bind specifically to at least one antigen (e.g., a protein, e.g., L1CAM/CD171). A binding agent often comprises at least one antigen binding portion (i.e., a binding portion). An antigen binding portion of a binding agent is that portion that binds specifically to an antigen. In certain embodiments a binding portion of a binding agent comprises or consists of a single polypeptide (e.g., single chain antibody). In some embodiments a binding portion of a binding agent comprises or consists of two polypeptides. In some embodiments a binding portion of a binding agent comprises or consists of 2, 3, 4 or more polypeptides. In some embodiments a binding agent comprises one or more structural portions (e.g., scaffolds, structural polypeptides, constant regions and/or framework regions). In some embodiments a binding agent, or binding portion thereof is attached to a substrate (e.g., a polymer, a non-organic material, silicon, a bead, particle, or the like).

In some embodiments a binding agent comprises an antibody, or a portion thereof (e.g., a binding portion thereof). In certain embodiments, a binding agent comprises or consists of an antibody, an antibody fragment and/or an antigen binding portion of an antibody (e.g., a binding fragment, i.e., a binding portion thereof). In some embodiments a binding agent is an antibody (e.g., a monoclonal antibody and/or a recombinant antibody).

The term “specifically binds” refers to a binding agent that binds to a target peptide in preference to binding other molecules or other peptides as determined by, for example, a suitable in vitro assay (e.g., an ELISA, immunoblot, flow cytometry, and the like). A specific binding interaction discriminates over non-specific binding interactions by about 2-fold or more, often about 10-fold or more, and sometimes about 100-fold or more, 1000-fold or more, 10,000-fold or more, 100,000-fold or more, or 1,000,000-fold or more.

Any suitable method can be used to detect and/or quantitate the presence, absence and/or amount of an miRNA in a neural-derived exosome fraction.

As used herein “amount” refers to a mass, a volume, and/or a concentration of a substance, such as a neural-derived exosome, molecule (such as a nucleic acid associated with a neural-enriched exosome fraction), drug, and the like. In some embodiments, presented herein are methods for detecting an amount, such as a mass, a volume and/or a concentration, of one or more nucleic acids associated with a neural-enriched exosome fraction. In some embodiments, presented herein are methods for determining an amount, such as a mass, a volume and/or a concentration, of one or more miRNAs associated with a neural-enriched exosome fraction. In some embodiments, presented herein are methods for measuring an amount, such as a mass, a volume and/or a concentration, of one or more miRNAs associated with a neural-enriched exosome fraction. In some embodiments, presented herein are methods for quantifying an amount, such as a mass, a volume and/or a concentration, of one or more miRNAs associated with a neural-enriched exosome fraction. In some embodiments, presented herein are methods for comparing an amount, such as a mass, a volume and/or a concentration, of one or more miRNAs associated with a neural-enriched exosome fraction with such an amount from a different sample containing such one or more miRNAs.

The term, “associated with” when used in the context of an exosome refers to one or more miRNAs that are present on, in or within, or expressed on, in, or within, an exosome.

In some embodiments, one or more miRNAs associated with neural-enriched exosome fraction comprises one or more microRNA (miRNA), and the like. In some embodiments, an miRNA associated with a neuro derived exosome is selected from the group consisting of miR-146a-5p, miR-199a-3p, miR-4454, miR-10b-5p, miR-29b-3p, miR-151a-3p, miR-151a-5p, and miR-199a-5p.

In some embodiments, a method presented herein detects or determines an amount of one or more miRNAs associated with a neural-derived exosome or neural-enriched exosome fraction. In some embodiments, a method herein can be conducted in vitro or ex vivo, such that an isolated sample is analyzed. In some embodiments, a method herein comprises determining the presence or amount of one or more miRNAs, such as miR-146a-5p, miR-199a-3p, miR-4454, miR-10b-5p, miR-29b-3p, miR-151a-3p, miR-151a-5p, and/or miR-199a-5p, that are associated with neural-enriched exo some fractions. In some embodiments, a method herein comprises determining the presence or amount of one or more miRNAs, such as miR-146a-5p, miR-199a-3p, miR-4454, miR-10b-5p, miR-29b-3p, miR-151a-3p, miR-151a-5p, and/or miR-199a-5p, that are associated with neural-enriched exosome fractions prepared from a sample obtained from a subject that has, or is suspected of having, a motor neuron disease. In some embodiments, a method herein comprises determining the presence or amount of one or more miRNAs, such as miR-146a-5p, miR-199a-3p, miR-4454, miR-10b-5p, miR-29b-3p, miR-151a-3p, miR-151a-5p, and/or miR-199a-5p, that are associated with neural-enriched exosome fractions prepared from a sample obtained from a control subject. Non-limiting examples of a control subject include healthy subjects, a subject that does not have a motor neuron disease and/or a subject that is not suspected of having a motor neuron disease.

In some embodiments, a method of identifying a subject who has, or is at risk of developing a motor neuron disease comprising: (a) determining a presence or amount of one or more micro-RNAs (miRNAs) in a sample obtained from the subject wherein the one or more miRNA are selected from the group consisting of miR-146a-5p, miR-199a-3p, miR-4454, miR-10b-5p, miR-29b-3p, miR-151a-3p, miR-151a-5p, and miR-199a-5p, and determining if the subject has, or is at risk of developing the motor neuron disease according to the presence or amount of the one or more miRNAs in the sample. In certain embodiments, the method comprises determine the presence or amount of two or more, three or more, four or more, five or more, six or more, seven or more or all eight of the miRNAs selected from the group consisting of miR-146a-5p, miR-199a-3p, miR-4454, miR-10b-5p, miR-29b-3p, miR-151a-3p, miR-151a-5p, and miR-199a-5p. In certain embodiments, the presence of four or more, five or more, six or more, seven or more or all eight of the miRNAs selected from the group consisting of miR-146a-5p, miR-199a-3p, miR-4454, miR-10b-5p, miR-29b-3p, miR-151a-3p, miR-151a-5p, and miR-199a-5p in, on or within a neuro-derived exosome obtained from a subject, indicates that the subject has or is at risk of having a motor neuron disease.

In some embodiments, a method herein comprises comparing an amount of one or more miRNAs obtained from neural-derived exosomes obtained from a sample from a control subject (e.g., a subject known to be free of a motor neuron disease) to an amount of one or more miRNAs obtained from neural-derived exosomes obtained from a sample from a test subject (e.g., a subject suspected of having a motor neuron disease). In some embodiments, the presence or absence of a motor neuron disease in a test subject is determined according to such a comparison. In some embodiments, a subject at risk of developing a motor neuron disease is identified according to such a comparison. In some embodiments, a comparison determines that the amount of one or more miRNAs associated with neural-derived exosomes obtained from a first subject are significantly lower, or significantly higher than those obtained from a control subject.

The term “significantly” as used throughout refers to a statically significant difference that can be determined using a suitable statistical method (e.g., a t-test). In some embodiments, a comparison determines that the amount of one or more miRNAs associated with a neural-enriched exosome fraction of a first subject are significantly higher than those of a control subject, thereby indicating that the first subject has a neurogenerative disease or has a high statistical likelihood of developing a neurogenerative disease.

In some embodiments, a comparison determines that the amount of one or more miRNAs associated with neural-derived exosomes obtained from a subject is from about 1.1-fold to about 20 fold higher or lower than a baseline amount of such one or more miRNAs, thereby indicating that the subject has a neurogenerative disease or has a statistical likelihood of developing a neurogenerative disease (i.e., is “at risk” of developing, e.g., a motor neuron disease such as, e.g., ALS). In some embodiments, a comparison determines that the amount of one or more miRNAs associated with a neural-enriched exosome fraction prepared from a sample obtained from a first subject is about 20-fold, about 19-fold, about 18-fold, about 17-fold, about 16-fold, about 15-fold, about 14-fold, about 13 fold, about 12 fold, about 11 fold, about 10.5 fold, about 10 fold, about 9.5 fold, about 9 fold, about 8.5 fold, about 8 fold, about 7.5 fold, about 7 fold, about 6.5 fold, about 6 fold, about 5.5 fold, about 5 fold, about 4.5 fold, about 4 fold, about 3.5 fold, about 3 fold, about 2.9 fold, about 2.8 fold, about 2.7 fold, about 2.6 fold, about 2.5 fold, about 2.4 fold, about 2.3 fold, about 2.2 fold, about 2 fold, about 1.9 fold, about 1.8 fold, about 1.7 fold, about 1.6 fold, about 1.5 fold, about 1.4 fold, about 1.3 fold, about 1.2, or about 1.1 fold higher or lower than the baseline amount of such one or more miRNAs, thereby indicating that the subject has a neurogenerative disease or has a statistical likelihood of developing a neurogenerative disease (i.e., is “at risk” of developing, e.g., a motor neuron disease such as, e.g., ALS).

In some embodiments, an amount of miR-146a-5p that is at least 1.1, at least 1.2, at least 1.3 or at least 1.4 fold higher that a base line amount of miR-146a-5p in, on and/or within neuro-derived exosomes obtained from a subject identifies that subject as having or at risk of developing a motor neuron disease. In some embodiments, an amount of miR-146a-5p that is at least 1.1, at least 1.2, at least 1.3 or at least 1.4 fold higher that a base line amount of miR-146a-5p in, on and/or within neuro-derived exosomes obtained from a subject identifies that subject as having or at risk of developing ALS.

In some embodiments, an amount of miR-199a-3p that is at least 1.4, at least 1.5, at least 1.6 or at least 2.0 fold higher that a base line amount of miR-199a-3p in, on and/or within neuro-derived exosomes obtained from a subject identifies that subject as having or at risk of developing a motor neuron disease. In some embodiments, an amount of miR-199a-3p that is at least 1.4, at least 1.5, at least 1.6 or at least 2.0 fold higher that a base line amount of miR-199a-3p in, on and/or within neuro-derived exosomes obtained from a subject identifies that subject as having or at risk of developing ALS.

In some embodiments, an amount of miR-4454 that is at least 1.4, at least 1.5, at least 1.6, at least 1.7, at least 1.8, or at least 2.0 fold lower that a base line amount of miR-4454 in, on and/or within neuro-derived exosomes obtained from a subject identifies that subject as having or at risk of developing a motor neuron disease. In some embodiments, an amount of miR-4454 that is at least 1.4, at least 1.5, at least 1.6, at least 1.7, at least 1.8, or at least 2.0 fold lower that a base line amount of miR-4454 in, on and/or within neuro-derived exosomes obtained from a subject identifies that subject as having or at risk of developing ALS.

In some embodiments, an amount of miR-10b-5p that is at least 2.0, at least 2.1, at least 2.5, at least 3.0, at least 4.0, or at least 5.0 fold lower that a base line amount of miR-10b-5p in, on and/or within neuro-derived exosomes obtained from a subject identifies that subject as having or at risk of developing a motor neuron disease. In some embodiments, an amount of miR-10b-5p that is at least 2.0, at least 2.1, at least 2.5, at least 3.0, at least 4.0, or at least 5.0 fold lower that a base line amount of miR-10b-5p in, on and/or within neuro-derived exosomes obtained from a subject identifies that subject as having or at risk of developing ALS.

In some embodiments, an amount of miR-29b-3p that is at least 1.4, at least 1.5, at least 1.6, at least 1.7, at least 1.8, or at least 2.0 fold lower that a base line amount of miR-29b-3p in, on and/or within neuro-derived exosomes obtained from a subject identifies that subject as having or at risk of developing a motor neuron disease. In some embodiments, an amount of miR-29b-3p that is at least 1.4, at least 1.5, at least 1.6, at least 1.7, at least 1.8, or at least 2.0 fold lower that a base line amount of miR-29b-3p in, on and/or within neuro-derived exosomes obtained from a subject identifies that subject as having or at risk of developing ALS.

In some embodiments, an amount of miR-151a-3p that is at least 1.4, at least 1.5, at least 1.6, at least 1.8, at least 2.0, or at least 2.2 fold higher that a base line amount of miR-151a-3p in, on and/or within neuro-derived exosomes obtained from a subject identifies that subject as having or at risk of developing a motor neuron disease. In some embodiments, an amount of miR-151a-3p that is at least 1.4, at least 1.5, at least 1.6, at least 1.8, at least 2.0, or at least 2.2 fold higher that a base line amount of miR-151a-3p in, on and/or within neuro-derived exosomes obtained from a subject identifies that subject as having or at risk of developing ALS.

In some embodiments, an amount of miR-151a-5p that is at least 1.4, at least 1.5, at least 1.6, at least 1.8, at least 2.0, or at least 2.2 fold higher that a base line amount of miR-151a-5p in, on and/or within neuro-derived exosomes obtained from a subject identifies that subject as having or at risk of developing a motor neuron disease. In some embodiments, an amount of miR-151a-5p that is at least 1.4, at least 1.5, at least 1.6, at least 1.8, at least 2.0, or at least 2.2 fold higher that a base line amount of miR-151a-5p in, on and/or within neuro-derived exosomes obtained from a subject identifies that subject as having or at risk of developing ALS.

In some embodiments, an amount of miR-199a-5p that is at least 1.9, at least 2.0, at least 2.4, at least 3.0, at least 3.5, or at least 4.2 fold higher that a base line amount of miR-199a-5p in, on and/or within neuro-derived exosomes obtained from a subject identifies that subject as having or at risk of developing a motor neuron disease. In some embodiments, an amount of miR-199a-5p that is at least 1.9, at least 2.0, at least 2.4, at least 3.0, at least 3.5, or at least 4.2 fold higher that a base line amount of miR-199a-5p in, on and/or within neuro-derived exosomes obtained from a subject identifies that subject as having or at risk of developing ALS.

In some embodiments, such a comparison determines that the amount of one or more of the miRNAs: miR-146a-5p; miR-199a-3p; miR-4454; miR-10b-5p; miR-29b-3p; miR-151a-3p; miR-151a-5p; and miR-199a-5p; associated with a neural-enriched exosome fraction prepared from a sample obtained from a subject, is about 20-fold to about 1.1 fold higher or lower than the baseline amount of such one or more miRNAs, thereby indicating that the subject has a neurogenerative disease or has a statistical likelihood of developing a neurogenerative disease (i.e., is “at risk” of developing, e.g., a motor neuron disease such as, e.g., ALS). In some embodiments, such a comparison determines that the amount of one or more of the miRNAs, miR-146a-5p; miR-199a-3p; miR-4454; miR-10b-5p; miR-29b-3p; miR-151a-3p; miR-151a-5p ;and miR-199a-5p, associated with a neural-enriched exosome fraction prepared from a sample obtained from a subject is about 20-fold, about 19-fold, about 18-fold, about 17-fold, about 16-fold, about 15-fold, about 14-fold, about 13 fold, about 12 fold, about 11 fold, about 10.5 fold, about 10 fold, about 9.5 fold, about 9 fold, about 8.5 fold, about 8 fold, about 7.5 fold, about 7 fold, about 6.5 fold, about 6 fold, about 5.5 fold, about 5 fold, about 4.5 fold, about 4 fold, about 3.5 fold, about 3 fold, about 2.9 fold, about 2.8 fold, about 2.7 fold, about 2.6 fold, about 2.5 fold, about 2.4 fold, about 2.3 fold, about 2.2 fold, about 2 fold, about 1.9 fold, about 1.8 fold, about 1.7 fold, about 1.6 fold, about 1.5 fold, about 1.4 fold, about 1.3 fold, about 1.2, or about 1.1 fold higher or lower than a baseline amount of such one or more nucleic, thereby indicating that the subject has a neurogenerative disease or has a statistical likelihood of developing a neurogenerative disease (i.e., is “at risk” of developing, e.g., a motor neuron disease such as, e.g., ALS.

The term “baseline amount” as used herein refers to an average, mean, or absolute amount of one or more miRNAs obtained from neuro-derived exosomes obtained from one or more suitable control subjects. For example, a control subject can be a subject that does not have a motor neuron disease. In certain embodiments, a control subject is a subject who does not have ALS. Typically such healthy subjects are young adults (e.g., within the ages of 18-30) that show no signs or symptoms of a motor neuron disease and/or have no family history of a motor neuron disease.

In some embodiments, a comparison determines that the amount of one or more miRNAs associated with a neural-enriched exosome fraction prepared from a sample obtained from a first subject is from about 20-fold to about 1.1 fold higher or lower than the amount of such one or more miRNAs determined from a neural-enriched exosome fraction prepared from a sample obtained from a control subject, thereby indicating that the first subject has a neurogenerative disease or has a statistical likelihood of developing a neurogenerative disease (i.e., is “at risk” of developing, e.g., a motor neuron disease such as, e.g., ALS). In some embodiments, a comparison determines that the amount of one or more miRNAs associated with a neural-enriched exosome fraction prepared from a sample obtained from a first subject is about 20-fold, about 19-fold, about 18-fold, about 17-fold, about 16-fold, about 15-fold, about 14-fold, about 13 fold, about 12 fold, about 11 fold, about 10.5 fold, about 10 fold, about 9.5 fold, about 9 fold, about 8.5 fold, about 8 fold, about 7.5 fold, about 7 fold, about 6.5 fold, about 6 fold, about 5.5 fold, about 5 fold, about 4.5 fold, about 4 fold, about 3.5 fold, about 3 fold, about 2.9 fold, about 2.8 fold, about 2.7 fold, about 2.6 fold, about 2.5 fold, about 2.4 fold, about 2.3 fold, about 2.2 fold, about 2 fold, about 1.9 fold, about 1.8 fold, about 1.7 fold, about 1.6 fold, about 1.5 fold, about 1.4 fold, about 1.3 fold, about 1.2, or about 1.1 fold higher or lower than the amount of such one or more miRNAs determined from a neural-enriched exosome fraction prepared from a sample obtained a control subject, thereby indicating that the first subject has a neurogenerative disease or has a statistical likelihood of developing a neurogenerative disease (i.e., is “at risk” of developing, e.g., a motor neuron disease such as, e.g., ALS. In some embodiments, such a comparison determines that the amount of one or more of the miRNAs: miR-146a-5p; miR-199a-3p; miR-4454; miR-10b-5p; miR-29b-3p; miR-151a-3p; miR-151a-5p ; and miR-199a-5p; associated with a neural-enriched exosome fraction prepared from a sample of a first subject, is about 20-fold to about 1.1 fold higher or lower than the amount of such one or more miRNAs determined from a neural-enriched exosome fraction obtained from of a control subject, thereby indicating that the first subject has a neurogenerative disease or has a statistical likelihood of developing a neurogenerative disease (i.e., is “at risk” of developing, e.g., a motor neuron disease such as, e.g., ALS). In some embodiments, such a comparison determines that the amount of one or more of the miRNAs: miR-146a-5p; miR-199a-3p; miR-4454; miR-10b-5p; miR-29b-3p; miR-151a-3p; miR-151a-5p ;and miR-199a-5p; associated with a neural-enriched exosome fraction prepared from a sample obtained from a first subject is about 20-fold, about 19-fold, about 18-fold, about 17-fold, about 16-fold, about 15-fold, about 14-fold, about 13 fold, about 12 fold, about 11 fold, about 10.5 fold, about 10 fold, about 9.5 fold, about 9 fold, about 8.5 fold, about 8 fold, about 7.5 fold, about 7 fold, about 6.5 fold, about 6 fold, about 5.5 fold, about 5 fold, about 4.5 fold, about 4 fold, about 3.5 fold, about 3 fold, about 2.9 fold, about 2.8 fold, about 2.7 fold, about 2.6 fold, about 2.5 fold, about 2.4 fold, about 2.3 fold, about 2.2 fold, about 2 fold, about 1.9 fold, about 1.8 fold, about 1.7 fold, about 1.6 fold, about 1.5 fold, about 1.4 fold, about 1.3 fold, about 1.2, or about 1.1 fold higher or lower than the amount of such one or more miRNAs determined from a neural-enriched exosome fraction obtained from a control subject, thereby indicating that the first subject has a neurogenerative disease or has a statistical likelihood of developing a neurogenerative disease (i.e., is “at risk” of developing, e.g., a motor neuron disease such as, e.g., ALS).

In some embodiments, the presence or absence of a motor neuron disease in a subject is determined according to an amount of one or more miRNAs that is associated with a neural-enriched exosome fraction prepared from a sample obtained from a subject. In some embodiments, an amount of at least 50 μL of miRNA, at least 100 μL of miRNA, at least 150 μL of miRNA, at least 200 μL of miRNA, at least 250 μL of miRNA, at least 300 μL of miRNA, at least 350 μL of miRNA, at least 400 μL of miRNA, at least 450 μL of miRNA, at least 500 μL of miRNA, at least 550 μL of miRNA, at least 600 μL of miRNA, at least 650 μL of miRNA, at least 700 μL of miRNA, at least 750 μL of miRNA, at least 800 μL of miRNA, at least 850 μL of miRNA, at least 900 μL of miRNA, at least 950 μL of miRNA, at least 1000 μL of miRNA, or more of miRNA derived from a neural-enriched exosome fraction prepared from a sample obtained from a subject indicates that the subject has a neurogenerative disease or has a statistical likelihood (i.e., is “at risk”) of developing a neurogenerative disease.

In some embodiments, an amount of: at least 1 pg/μL of miRNA, at least 2 pg/μL of miRNA, at 3 pg/μL of miRNA, at least 4 pg/μL of miRNA, at least 5 pg/μL of miRNA, at least 10 pg/μL of miRNA, at least 15 pg/μL of miRNA, at least 20 pg/μL of miRNA, at least 25 pg/μL of miRNA, at least 30 pg/μL of miRNA, at least 35 pg/μL of miRNA, at least 40 pg/μL of miRNA, at least 45 pg/μL of miRNA, 50 pg/μL of miRNA, at least 100 pg/μL of miRNA, at least 150 pg/μL of miRNA, at least 200 pg/μL of miRNA, at least 250 pg/μL of miRNA, at least 300 pg/μL of miRNA, at least 350 pg/μL of miRNA, at least 400 pg/μL of miRNA, at least 450 pg/μL of miRNA, at least 500 pg/μL of miRNA, at least 550 pg/μL of miRNA, at least 600 pg/μL of miRNA, at least 650 pg/μL of miRNA, at least 700 pg/μL of miRNA, at least 750 μL of miRNA, at least 800 μL of miRNA, at least 850 μL of miRNA, at least 900 μL of miRNA, at least 950 μL of miRNA, at least 1000 μL of miRNA, or more of miRNA, wherein the miRNA comprises one or more of miR-146a-5p, miR-199a-3p, miR-4454, miR-10b-5p, miR-29b-3p, miR-151a-3p, miR-151a-5p, and miR-199a-5p derived from exosome indicates that a subject has a neurogenerative disease or has a statistical likelihood (i.e., is “at risk”) of developing a neurogenerative disease.

In some embodiments, methods are provided for monitoring the progression of a motor neuron disease in a subject. In some embodiments, such a method comprises a) preparing a neural-enriched exosome fraction from a sample obtained from the subject; (b) determining an amount of one or more miRNAs from the neural-enriched exosome fraction, such as one or more miRNAs selected from the group consisting of miR-146a-5p, miR-199a-3p, miR-4454, miR-10b-5p, miR-29b-3p, miR-151a-3p, miR-151a-5p, and miR-199a-5p associated with the neural-enriched exosome fraction obtained from the subject; and (c) comparing the amount of the one or more miRNAs determined in step (b) to a baseline amount of the one or more miRNAs.

In some embodiments, methods are provided for monitoring a response to treatment of a motor neuron disease in a subject. In some embodiments, such a method comprises a) preparing a neural-enriched exosome fraction from a sample obtained from the subject after treatment of the subject has commenced; (b) determining an amount of one or more miRNAs from the neural-enriched exosome fraction, such as one or more miRNAs selected from the group consisting of miR-146a-5p, miR-199a-3p, miR-4454, miR-10b-5p, miR-29b-3p, miR-151a-3p, miR-151a-5p, and miR-199a-5p associated with the neural-enriched exosome fraction; and (c) comparing the amount of the one or more miRNAs determined in step (b) to a baseline amount of the one or more miRNAs. In some embodiments, a difference between an amount of one or more miRNAs from the a neural-enriched exosome fraction obtained from a subject after treatment of the subject has commenced and a baseline amount that is less that the difference obtained at an earlier point in time indicates that the subject has responded favorably to the treatment. In some embodiments, a difference between an amount of one or more miRNAs from neural-enriched exosome fraction obtained from a subject and a baseline amount that is greater that the difference obtained at an earlier point in time indicates that the subject has not responded favorably to the treatment.

In some embodiments, the baseline amount may be an amount considered ‘normal’ for the particular miRNA (e.g., an average amount for age-matched individuals not diagnosed with the motor neuron disease), or may be a historical reference amount for the particular subject (e.g., a baseline amount that was obtained from a sample derived from the same subject, but at an earlier point in time). Quantitative baseline amounts which are determined contemporaneously (e.g., a reference value that is derived from a pool of samples including the sample being tested) are also contemplated. Accordingly, in some embodiments, methods are provided for monitoring progression of a motor neuron disease in a subject by obtaining a quantitative measured amount for one or more miRNAs associated with neuronal-enriched exosomes obtained from a sample and comparing such measured value to a baseline amount. In some embodiments, a difference between an amount of one or more miRNAs from a neural-enriched exosome fraction obtained from a subject and a baseline amount that is less that the difference obtained at an earlier point in time indicates that the progression of the disease has diminished. In some embodiments, a difference between an amount of one or more miRNAs from a neural-enriched exosome fraction obtained from a subject and a baseline amount that is greater that the difference obtained at an earlier point in time indicates that the progression of the disease has increased.

Administration

Any suitable method of administering a treatment or drug to a subject can be used. Any suitable formulation and/or route of administration can be used for administration of a treatment or drug disclosed herein (e.g., see Fingl et al. 1975, in “The Pharmacological Basis of Therapeutics”, which is incorporated herein by reference in its entirety). A suitable formulation and/or route of administration can be chosen by a medical professional (e.g., a physician) in view of, for example, a subject's disease, condition, symptoms, weight, age, and/or general health. Non-limiting examples of routes of administration include topical or local (e.g., transdermally or cutaneously, (e.g., on the skin or epidermis), in or on the eye, intranasally, transmucosally, in the ear, inside the ear (e.g., behind the ear drum)), enteral (e.g., delivered through the gastrointestinal tract, e.g., orally (e.g., as a tablet, capsule, granule, liquid, emulsification, lozenge, or combination thereof), sublingual, by gastric feeding tube, rectally, and the like), by parenteral administration (e.g., parenterally, e.g., intravenously, intra-arterially, intramuscularly, intraperitoneally, intradermally, subcutaneously, intracavity, intracranial, intra-articular, into a joint space, intracardiac (into the heart), intracavernous injection, intralesional (into a skin lesion), intraosseous infusion (into the bone marrow), intrathecal (into the spinal canal), intrauterine, intravaginal, intravesical infusion, intravitreal), the like or combinations thereof.

In some embodiments administering a drug to a subject comprises provided the drug to the subject, for example for self-administration or for administration to the subject by another (e.g., by a non-medical professional). As another example, a drug can be provided as an instruction written by a medical practitioner that authorizes a patient to be provided a drug or treatment described herein (e.g., a prescription). In yet another example, a drug can be provided to a subject where the subject self-administers a composition orally, intravenously or by way of an inhaler, for example.

Alternately, one can administer a drug in a local rather than systemic manner, for example, via direct application to the skin, mucous membrane or region of interest for treating, including using a depot or sustained release formulation.

In certain embodiments a drug is administered alone (e.g., as a single active ingredient (AI) or, e.g., as a single active pharmaceutical ingredient (API)). In other embodiments, a drug is administered in combination with one or more additional AIs/APIs, for example, as two separate compositions or as a single composition where the one or more additional AIs/APIs are mixed or formulated together with a drug in a pharmaceutical composition.

In some embodiments, an amount of a motor neuron disease drug administered to a subject is a therapeutically effective amount. In some embodiments, a therapeutically effective amount of a drug is an amount needed to obtain an effective therapeutic outcome. In certain embodiments, a therapeutically effective amount of a drug is an amount sufficient to treat, reduce the severity of, inhibit or delay the onset of, mitigate and/or alleviate one or more symptoms of a motor neuron disease. Determination of a therapeutically effective amount is well within the capability of those skilled in the art, especially in light of the detailed disclosure provided herein.

In certain embodiments, a therapeutically effective amount is an amount high enough to provide an effective therapeutic effect (e.g., a beneficial therapeutic effect) and an amount low enough to minimize unwanted adverse reactions. Accordingly, in certain embodiments, a therapeutically effective amount of a drug may vary from subject to subject, often depending on age, weight, general health condition of a subject, severity of a condition being treated and/or a particular combination of drugs administered to a subject. Thus, in some embodiments, a therapeutically effective amount is determined empirically. Accordingly, in certain embodiments, a therapeutically effective amount of a drug that is administered to a subject can be determined by one of ordinary skill in the art based on amounts found effective in animal or clinical studies, a physician's experience, and/or suggested dose ranges or dosing guidelines.

In certain embodiments, a therapeutically effective amount of L-serine or a composition disclosed herein comprises one or more doses (administered to a subject) comprising at least 0.1 mg/kg, at least 5 mg/kg, at least 10 mg/kg, at least 15 mg/kg, at least 20 mg/kg, at least 25 mg/kg, at least 50 mg/kg, at least 100 mg/kg, at least 250 mg/kg, at least 500 mg/kg, at least 1000 mg/kg, at least 5000 mg/kg, or at least 7500 mg/kg of L-serine, or a salt, a precursor, derivative or conjugate thereof, per kg body weight of a subject.

In some embodiments administering a therapeutically effective amount of a motor neuron disease drug or composition disclosed herein comprises administering a suitable dose hourly, every two hours, every 4 hours, every 6 hours, every 8 hours, or every 12 hours. In certain embodiments, a motor neuron disease drug can be administered at least one, at least two, at least three, at least four, at least five, or at least six times per day, e.g., 1 to 12 times per day, 1 to 8 times per day, or 1 to 4 times per day per day. In certain embodiments, a motor neuron disease drug disclosed herein can be administered once, twice, 3 times, 4 times, 5 times, 6 times, 7 times, 8 times, 9 times, 10 times, 11 times, or 12 times per day. A motor neuron disease drug may be administered in a single dosage form or one or more dosage forms. A daily dose can be achieved in the form of a single dose or in the form of a plurality of partial doses.

A motor neuron disease drug disclosed herein can be administered on a daily basis or on a schedule containing days where dosing does not take place. For example, dosing may take place every other day, or dosing may take place for 2, 3, 4, or 5 consecutive days of a week, then be followed by from 1 to 5 non-dosing days.

A motor neuron disease drug can be administered for at least a day, at least two days, at least three days, at least four days, at least five days, at least a week, at least two weeks, at least three weeks, at least a month, at least two months, at least three months, at least six months, at least a year, at least two years, or more, or for any extended duration to further improve, maintain, or retain therapeutic efficacy. In certain embodiments, a motor neuron disease drug is administered for a duration of 1 week to 10 years or more. In some embodiments administering a therapeutically effective amount of a drug, or a pharmaceutical composition comprising a drug, comprises administering a suitable dose at a frequency or interval as needed to obtain an effective therapeutic outcome. In some embodiments administering a therapeutically effective amount of a drug or a pharmaceutical composition disclosed herein comprises administering a suitable dose hourly, every two hours, every 4 hours, every 6 hours, three times a day, twice a day, once a day, six times a week, five times a week, four times a week, three times a week, twice a week, weekly, at combinations thereof, and/or at regular or irregular intervals thereof, and/or simply at a frequency or interval as needed or recommended by a medical professional. In some embodiments, a therapeutically effective amount of a drug or a pharmaceutical composition comprising a therapeutically effective amount of drug is administered continuously by, for example by intravenous administration.

### EXAMPLES

### Example 1—Preparation and Quantification Neural-Enriched Exosome Fractions

Venous blood was collected in K2 EDTA tubes and centrifuged for 15 min at 2,000×g at 4° C. Blood plasma was removed and divided into 0.5 ml aliquots and immediately frozen and stored at −80° C. Time between blood collection and freezing was less than 1 hour. One half ml of plasma was incubated with 0.15 μl thromboplastin-D at room temperature for 60 min. Exosomes were prepared as per Mustapic, M, et al., (2017) (Frontiers in Neuroscience 11:278), with some modification. Briefly, 0.15 μl of Dulbecco balanced salt solution (DBS⁻² calcium- and magnesium-free) was added along with three times the recommended concentrations of Halt protease inhibitor cocktail and Halt phosphatase inhibitor cocktail. The mixture was then centrifuged at 1500×g for 20 min. ExoQuick solution (134 μl, SBI) was then added to precipitate total exosomes and the solution incubated at 4° C. overnight. The sample was centrifuged at 1500 x g for 30 min. and the supernatant discarded. The pellet was resuspended in 250 μl of DBS⁻² with the protease and phosphatase inhibitors indicated above, followed by centrifugation at 1500×g for 5 min. Generation of a neural-enriched exosome fraction was accomplished by the addition of 2 μg of biotinylated mouse anti-human CD171 [L1 cell adhesion molecule (L1CAM)] antibody (clone 5G3) in 50 μl of 3% bovine serum albumin (BSA) for 60 min. at 2° C. Streptavidin-agarose resin (˜25 μl) and 3% BSA (50 μl) was then added and the mixture was centrifuged at 1500 x g for 5 min. The supernatant was removed and labeled as “Total-Neural” (i.e., Total minus Neural) exosome fraction. The pellet containing the neural-enriched exosome fraction was then suspended in 50 μl of 0.05 M glycine-HCL (pH 3.0) and vortexed for 10 seconds, followed by the addition of 0.45 ml of DBS⁻² containing 3% BSA and inhibitor cocktails. This mixture was incubated for 10 min. at 37° C. with vortex-mixing. After centrifugation (1500 x g for 5 min) the supernatant was transferred to a new Eppendorf tube followed by the addition of 5 μl of 1 M Tris-HCL (pH 8.0). The neural-enriched exosome fraction was lysed with 0.40 ml of M-PER mammalian protein extraction reagent containing protease and phosphatase inhibitors. Protein concentrations were assessed with Qubit 3 Fluorometer. Samples were stored at −80° C. pending further analysis.

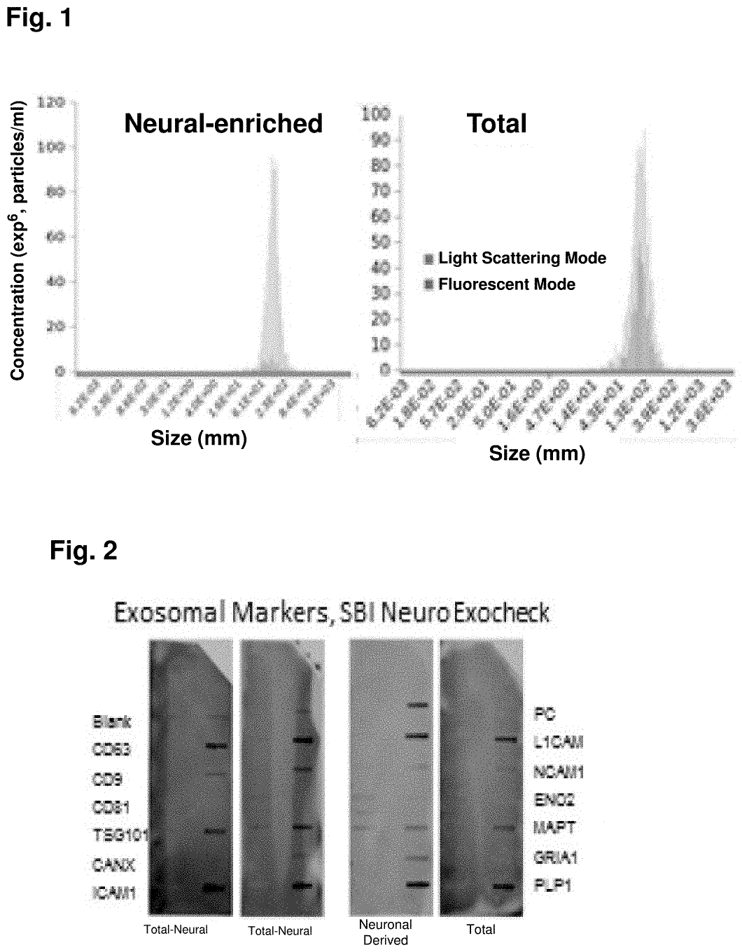

Fluorescence NTA and Light Scatter NTA were used to quantitate the amount of exosome particles in the neural enriched exosome fraction. Table 1 and FIG. 1 shows the results of these assays.

TABLE 1

| | Light Scatter NTA | | Fluorscence NTA | |
|---|---|---|---|---|
| | (particles/ml) | | (particles/ml) | |
| Exosome | | Peak Size | | Peak Size |
| Type | Particles/ml | (nm) | Particles/ml | (nm) |
| Neural | 47 billion | 124 | 6.1 billion | 101 |
| Neural | 62 billion | 126 | 37 billion | 101 |
| Total | 630 billion | 126 | 180 billion | 129 |
| Total | 440 billion | 117 | 210 billion | 117 |

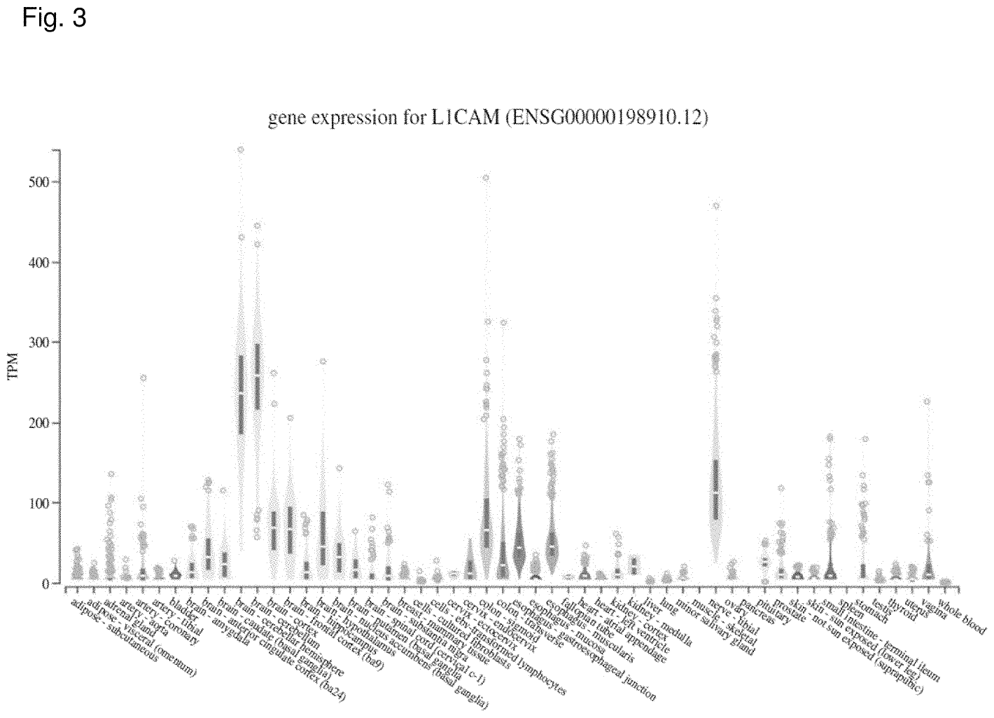

Neural enriched exosome fraction preparation was analyzed for the presence or absence of contaminating cell markers, neural exosome markers, cell adhesion molecules, and tetraspanins indicated in FIG. 2. Table 2 shows the amounts of exosomes expressing CD81 (Tetraspanin-28) determined by ELISA (SBI-ExoElisa-ultra CD81).

TABLE 2

| SAMPLE | Billions of exosomes/ug Protein |
|---|---|
| Neural-enriched | >7.6 |
| Total | >3.4 |
| Total minus Neural-enriched | 1.2-8.5 |

### Example 2—Detection and Quantitation of messenger RNA In Neural-Enriched Exosome Fractions

An RT2 Profiler™ PCR Array Human Unfolded Protein Array (QIAGEN, product number 330231, Cat. No. PAHS-089Z) was used, with a pre-amplification step, to profile the amount of messenger RNA in neural-enriched exo some fractions to determine, in part, if neural-enriched exosome fractions could provide enough messenger RNA for cDNA synthesis. Briefly, total RNA was extracted from 50 μL of total neural-enriched exosome fractions using the SeraMir kit from System Biosciences according to the manufacturer's instructions with some modifications, as follows. Following lysis of neural-enriched exosome fractions, RNA was eluted into 2×15 μL pre-warmed (37° C.) Elution Buffer using the following protocol; 15 μL Elution Buffer was added directly to the membrane then the tubes spun at 2000 rpm to load the membrane. The speed was then increased to 13,000 rpm for 1 min to elute the exoRNA. This elution step was repeated once more to result in a final volume of exoRNA of ˜30 μL. ExoRNA was pre-amplified using the QIAGEN RT2 PreAMP cDNA Synthesis Kit to increase the likelihood of seeing a signal on the array, in accordance with the manufacturer's instructions. Briefly, genomic DNA was eliminated from each 8 μL RNA sample using the genomic DNA elimination mix and cDNA was synthesized using the QIAGEN First Strand cDNA Synthesis kit, in accordance with the manufacturer's instructions, which includes a spiked-in control (P2) to monitor reverse transcription efficiency.

Real-time PCR using the RT2 Profiler PCR arrays: The pre-amplification reaction was mixed with 2×SYBR green and loaded onto the RT2 Profiler™ PCR Array Human Unfolded Protein Array, then real-time PCR run with the following conditions; activation 10 min 95° C., then 40 cycles of 15 secs, 95° C., and 1 min, 60° C. with data collection at step 2. A melt-curve was included. Data was exported to an Excel spreadsheet, then uploaded to the QIAGEN portal for analysis. All quality control markers passed, however, of the 81 genes on the array, only 28 amplified at or near 35 cycles, which constitutes the threshold limit for a meaningful signal (Table 3 & Table 4, below).

TABLE 3

| UniGene | GeneBank | | | |
|---|---|---|---|---|
| ID | ID | Symbol | Description | Ct |
| Hs.42853 | NM_004381 | ATF6B | Activating transcription | 33.48 |
| | | | factor 6 beta | |
| Hs.624291 | NM_004324 | BAX | BCL2-associated X protein | 34.52 |
| Hs.522110 | NM_006368 | CREB3 | CAMP responsive element | 33.55 |
| | | | binding protein 3 | |
| Hs.241576 | NM_024295 | DERL1 | Den-like domain family, | 33.66 |
| | | | member 1 | |
| Hs.77768 | NM_006736 | DNAJB2 | DnaJ (Hsp40) homolog, | 34.9 |
| | | | subfamily B, member 2 | |
| Hs.172847 | NM_005528 | DNAJC4 | DnaJ (Hsp40) homolog, | 34.84 |
| | | | subfamily C, member 4 | |
| Hs.224616 | NM_014674 | EDEM1 | ER degradation enhancer, | 31.9 |
| | | | mannosidase alpha-like 1 | |
| Hs.655782 | NM_032025 | EIF2A | Eukaryotic translation | 32.36 |
| | | | initiation factor 2A, 65 kDa | |
| Hs.592041 | NM_033266 | ERN2 | Endoplasmic reticulum to | 31.23 |
| | | | nucleus signaling 2 | |
| Hs.154023 | NM_015051 | ERP44 | Endoplasmic reticulum | 31.06 |
| | | | protein 44 | |
| Hs.693779 | NM_198141 | GANC | Glucosidase, alpha; neutral | 32.15 |
| | | | C | |
| Hs.115721 | NM_013247 | HTRA2 | HtrA serine peptidase 2 | 33.5 |
| Hs.7089 | NM_016133 | INSIG2 | Insulin induced gene 2 | 30.8 |
| Hs.443490 | NM_015884 | MBTPS2 | Membrane-bound | 31.33 |
| | | | transcription factor | |
| | | | peptidase, site 2 | |
| Hs.527861 | NM_006812 | OS9 | Osteosarcoma amplified 9, | 34.57 |
| | | | endoplasmic reticulum lectin | |
| Hs.655327 | NM_002624 | PFDN5 | Prefoldin subunit 5 | 29.11 |
| Hs.356331 | NM_021130 | PPIA | Peptidylprolyl isomerase A | 33.55 |
| | | | (cyclophilin A) | |
| Hs.610830 | NM_002743 | PRKCSH | Protein kinase C substrate | 31.55 |
| | | | 80K-H | |
| Hs.718462 | NM_006913 | RNF5 | Ring finger protein 5 | 33.91 |
| Hs.26904 | NM_007214 | SEC63 | SEC63 homolog (S. | 34.38 |
| | | | cerevisiae) | |
| Hs.32148 | NM_203472 | SELS | Selenoprotein S | 31.84 |
| Hs.363137 | NM_030752 | TCP1 | T-complex 1 | 34.53 |
| Hs.520640 | NM_001101 | ACTB | Actin, beta | 28.38 |
| Hs.534255 | NM_004048 | B2M | Beta-2-microglobulin | 27.66 |
| Hs.592355 | NM_002046 | GAPDH | Glyceraldehyde-3-phosphate | 30.51 |
| | | | dehydrogenase | |
| Hs.546285 | NM_001002 | RPLP0 | Ribosomal protein, large, P0 | 28.26 |

Of the 81 genes on the QIAGEN RT2 Profiler Human UPR array, 26 amplified at or near 35 cycles (see Table 3), which constituted the threshold limit for a meaningful signal. This suggested that neural-enriched exosome fractions do not contain sufficient full-length messenger RNA to facilitate effective quantitation by SYBR green qPCR.

TABLE 4

| Abbreviation | Description | CT | Mean | SD |
|---|---|---|---|---|
| GDC | genomic DNA | 36.31, 36.03, 36.50 | 36.28 | 0.24 |
| | amplification control | | | |
| RTC | cDNA synthesis | 27.52, 26.97, 27.33, | 27.23 | 0.26 |
| | reaction control | 27.04, 27.07, 27.41, | | |
| | | 27.04, 27.12, 27.41 | | |
| PPC | PCR positive control | 20.64, 21.03, 21.00, | 20.93 | 0.17 |
| | | 20.54, 21.01, 20.96, | | |
| | | 20.87, 21.15, 21.03 | | |

Table 4, above, provides quality control parameters included on the RT2 Profiler Array for Human Unfolded Protein Response were all met for total RNA extracted from exosomes. GDC=genomic DNA contamination; a CT≥35 cycles indicates GDNA is not contributing to the signal. RTC=reverse transcription control; an artificial mRNA with a poly-A tail that is not homologous to any mammalian or bacterial sequence is pre-loaded into the primer buffer of the RT2 First Strand cDNA synthesis kit and reverse transcribed along with the messages in the samples. The RTC detecting this sequence determines if the reverse transcription efficiency was similar for all samples. PPC=positive PCR control; the PPC wells contain a small amount of DNA with another artificial sequence (not homologous to the RTC) and primers designed to amplify this sequence. Testing and verification of the PPC defined that the PPC CT range should always yield CT values within a specific range (20+/<2) and be consistent within an array and between arrays. If the PPC CT values are not within this range, then the PCR itself was likely negatively impacted.

### Example 3—An miRNA Fingerprint of miRNA Neural-derived Exosomes for Amyotrophic Lateral Sclerosis/motor Neuron Disease

Motor neuron diseases such as Alzheimer's disease (AD), Parkinson's disease (PD) and amyotrophic lateral sclerosis/motor neuron disease (ALS/MND) continue to provide challenges for diagnosis since validated, clinically useful diagnostic neural-derived exosomes are currently unavailable. Rapid diagnosis and intervention in the disease process could be beneficial in slowing motor neuron disease progression as well as facilitating the testing of new therapies. In ALS/MND, the average time from diagnosis to death is typically short (2-5 years) and it is not unusual for patients to wait a year before receiving a diagnosis. Since disease progression correlates with motor neuron loss, early intervention could be critical for the development of new effective drug therapies. Current investigative drugs suggest some hope to reduce the rate of ALS/MND disease progression; however, the discovery of neural-derived exosomes would be a tremendous asset to these efforts.

To date, an ALS/MND diagnosis is based on clinical features with the elimination of alternative diagnoses and supporting data retrieved from electromyograms, nerve conduction studies, muscle biopsies, magnetic field imaging and biofluid analysis. The search for ALS/MND neural-derived exosomes useful for diagnosis, prognosis and analysis of drug efficacy includes a variety of molecules found in biofluids and other techniques including: heavy and light chain neurofilaments, TAR DNA-binding protein 43 (TDP-43), a lipid peroxidation product (4-hydroxy-2,3-nonenal), a urinary neurotrophin receptor p75 extracellular domain, cystatin C, mRNA, miRNA, extracellular glutamate, markers of inflammation, microglial activation, electrical impedance myography, rate of disease progression, spinal cord imaging and others. Thus far, none of these neural-derived exosomes have been sufficiently validated to be incorporated into the clinical standard of care.

Neural-derived exosome exploration has increased in recent years due to advances in cellular biology. The discovery of intercellular communication through exosomes has initiated new avenues for neural-derived exosome exploration. Exosomes are characterized as lipid membrane vesicles of endosomal origin of 30-200 nm in size, that contain a heterogeneous mix of messenger RNA (mRNA), microRNA (miRNA), transfer RNA (tRNA), Y RNA, small non-coding RNA (sRNA), DNA, lipids and proteins. Extracellular vesicles (EVs) are a more inclusive term for nucleus-absent, lipid bilayer particles, including exosomes, that are naturally released from the cell. EVs released into the extracellular matrix and taken up by adjacent cells impact cellular function of the recipient cells and possess both therapeutic and pathogenic potential. EVs are thought to be expelled from all cell types and can be isolated from diverse biological fluids including cerebrospinal fluid (CSF), plasma, serum, breast milk, lymph, bile and saliva. EVs are remarkably stable in bodily fluids, providing protection for their molecular cargo from enzymatic breakdown. This stability combined with their availability in easily obtainable biological fluids make them of interest as potential reservoirs for disease neural-derived exosomes, which in turn could be potentially useful for assessing the efficacy of therapeutic interventions.

In parallel with current research on EVs, the investigation of miRNA has independently shown promise in the quest for neural-derived exosomes. Since miRNA are post-transcriptional regulators of gene expression, mediated via suppression of the translation of mRNAs or degradation of target mRNAs, they transmit executable instructions. They have been identified as potential neural-derived exosomes in many fields including cancer, AD, systemic lupus erythematous, traumatic brain injury, cardiovascular disease, PD, multiple sclerosis and diabetes. Since miRNA are found as cargo within EVs and the lipid membrane surrounding EVs protects the miRNA from enzymatic degradation, there is good rational for examining miRNA extracted from isolated EVs. Added to this the potential for selectively enriching EVs by subtypes based on the specific protein surface markers, these techniques can be targeted and potentially produce reliable, stable disease markers.

In this study, eight miRNA sequences were identified from neural-derived exosome extractions of blood plasma that consistently and significantly differentiate ALS/MND patients from healthy controls. Since the composition of blood extractions likely includes small molecules and some other EV subtypes, we choose here to use the more generic term of EV. Exploiting cell-specific protein markers, we isolated a neural-enriched sub-population of extracellular vesicles (NEE) as a mechanism for analyzing neural-specific cargo. This technique generates a pool of NEE that are much more specific, reliable, and repeatable than other sources of neural-derived exosomes.

We compared the miRNA cargo from NEE in order to examine differential expression of miRNA between plasma samples from healthy controls and ALS/MND patients.

### Methods

Forty total plasma samples were analyzed in two independent experiments performed using identical criteria. Ten plasma samples were obtained from a blood draw of ALS/MND patients at the time they enrolled in a Phase IIa human clinical trial (NCT03580616). ALS/MND patients were compared with 10 healthy control plasma samples (Innovative Research Inc., Novi, Mich., USA). Following this experiment, a second cohort of 10 ALS/MND patients and 10 controls were independently analyzed using the same methods and the results compared for repeatability. ALS/MND patients met the following criteria: (1) diagnosis of probable or definite ALS/MND based on the El Escorial criteria [34] within the last 3 years prior to study enrolment; (2) ALSFRS-R score>25 and a FVC score≥60% predicted; (3) age≥18 years old. Prescription medications of both Riluzole and Endaravone/Radicava were allowed as long as the patient had taken these FDA-approved drugs for three months prior to trial enrolment and maintained a stable dose throughout the trial. None of the ALS/MND patients had a diagnosis or previous history of ischemic stroke, brain tumour, uncontrolled diabetes, renal insufficiency or severe hypertension. Severe hypertension (asymptomatic or hypertensive urgency) was defined as severely elevated blood pressure (180 mm Hg or more systolic, or 110 mm Hg or more diastolic) without acute target organ injury. None of the ALS/MND patients had a diagnosis or previous history of peripheral neuropathy or any other comorbid progressive motor neuron disease such as AD, PD, Lewy Body Disease, Pick's Disease, Huntington's Disease or Progressive Supranuclear Palsy. None of the ALS/MND patients were undergoing any chemotherapy or radiation therapy for any cancer. None were pregnant women or women who were breast feeding a child. Genetic analysis was not performed on these patients as it was outside the scope of this study. In addition to the 20 control patients identified above, an additional four healthy control plasma samples were used in a pilot study to determine if there was sufficient material for RNA extraction, NGS and qPCR (Qiagen Genomic Services). The pilot study validation also compared miRNA content of different extraction fractions to examine them for distinct signatures.

### Plasma Extraction

Venous blood was drawn into K2 EDTA tubes followed by immediate centrifugation at 2000×g for 15 min (4° C.). The plasma was removed prior to being frozen at −80° C. Time between blood collection and freezing was less than 1 h.

### EV Extraction

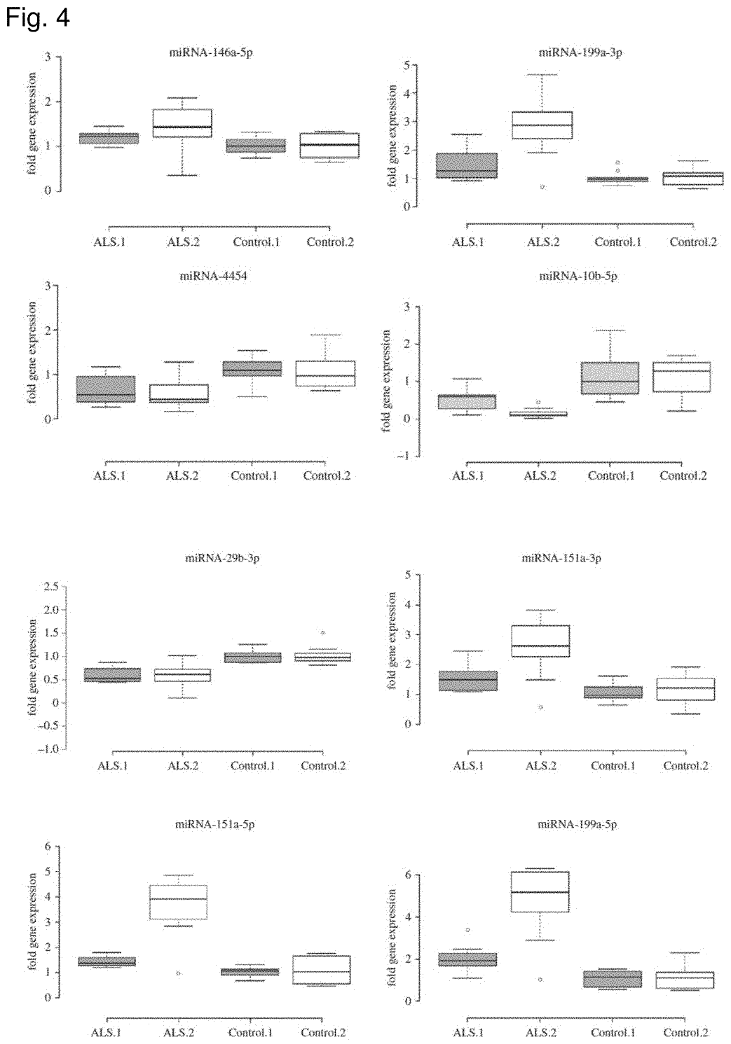

Plasma samples were thawed on ice or at 4° C., treated with thrombin to remove fibrinogen, and the EVs were precipitated using polyethylene glycol (SBI ExoQuick, Cat. No. EXOQ5TM-1, System Biosciences Inc, Palo Alto, Calif., USA). L1 cell adhesion molecule (L1CAM) antibodies were used to selectively separate NEE (Mustapic M, et al., (2017) Front. Neurosci. 11, 278.). Since L1CAM is a neural adhesion molecule and is highly expressed in brain and neural tissues (FIG. 3), this step creates a neural-enriched fraction of EVs with characteristics consistent with exosomes. In brief, 500 μl of plasma was incubated with 15 μl thrombin at room temperature for 30 min. To this 485 μl of sterile Dulbecco's phosphate-buffered saline balanced salt solution (DBS-2 calcium- and magnesium-free, Caisson Labs PBL01, Smithfield, Utah, USA) mixed with three times the recommended concentrations of Halt protease inhibitor cocktail (Cat. No. 78429, Thermo Fisher Scientific, Waltham, Mass., USA) and Halt phosphatase inhibitor cocktail (Cat. No. 78426, Thermo Fisher Scientific,Waltham, Mass., USA) was added. The mixture was then centrifuged at 4500 g for 20 min (4° C.). To the supernatant, ExoQuick precipitation solution (252 μl, Cat. No. SBI EXOQ20A-1, System Biosciences Inc, Palo Alto, Calif., USA) was then added to precipitate extracellular vesicles and the solution was incubated at 4° C. for 1 h. The sample was centrifuged at 1500 g for 20 min (4° C.), and the supernatant discarded. The pellet was resuspended in 500 μl of ultra-pure water that contained the 3×protease and phosphatase inhibitors, vortexed gently and then placed on a rotating mixer overnight. This fraction represents the total extracellular vesicle extraction.

### Neural-enriched EV Extraction

Enrichment of neural-enriched EVs was accomplished by the addition of 4 μg of mouse anti-human CD171 (L1 cell adhesion molecule (L1CAM) neural adhesion protein) monoclonal antibody [Cat. No. eBIO5G3 (5G3), (13-1719-82), Biotin, eBioscience™ Antibodies, Thermo Fisher Scientific, Waltham, Mass., USA) in 50 μl of 3% bovine serum albumin (BSA) (Cat. No. 37525, Block BSA 10× in PBS, Thermo Fisher Scientific, Waltham, Mass., USA) for 60 min at 4° C. on a rotating mixer. To this solution, we then added 15 μl of streptavidin-agarose resin (Cat. No. 53116, Pierce Streptavidin Plus UltraLink Resin, Thermo Fisher Scientific, Waltham, Mass., USA) plus 25 μlof 3% BSA. This mixture was incubated at 4° C. on a rotating mixer for 30 min followed by the addition of 4 μl of ultra-pure 1 M TRIS-HCl pH 8.0 (Cat. No. 15568025, Thermo Fisher Scientific, Waltham, Mass., USA) to adjust pH to 7.0. The mixture was then centrifuged at 200 g for 10 min (4° C.). The supernatant fraction represents the total heterogeneous extracellular vesicle population minus the extracellular vesicles with L1CAM neural surface proteins, a fraction which we designate as T-N. The pellet containing the neural-enriched EVs (NEE) was then suspended in 200 μl of 0.1 M glycine-HCl (pH 2.5) and the solution was strongly vortexed and centrifuged at 4500 g for 5 min (4° C.). The supernatant was recovered and neutralized with 15 μl 1 M TRIS-HCl pH 8.0. The T-N and the NEE fractions were tested for protein content using Molecular Probes Quant iT Qubit Protein Assay Kit (Cat. No. Q33211, Thermo Fisher Scientific, Waltham, Mass., USA) using a Qubit 3 fluorometer (Cat. No. Q33216, Invitrogen, Thermo Fisher Scientific, Waltham, Mass., USA) and frozen in aliquots (−80° C.).

### Characterization of Extracellular Vesicles

EVs were characterized using a ZetaView® NTA System (Particle Metrix Inc. Henderson, Nev., USA) in both light and fluorescence modes (Cat. No. EXONTA110A-1, System Biosciences Inc., Palo Alto, Calif., USA). Further characterization of surface proteins was conducted using the following kits according to manufacturer's instructions: human CD81 ELISA Kit (Sandwich ELISA) (Cat. No. LS-F55938, LSBio Seattle, Wash., USA); human CD63 ELISA Kit (Sandwich ELISA) (Cat. No. LS-F7104, LSBio Seattle, Wash., USA) and Exo-Check™ exosome antibody array (Neuro) (Cat. No. EXORAY500A-8, System Biosciences Inc., Palo Alto, Calif., USA).

RNA Extraction from EVs, Library Construction and Next-generation Sequencing

RNA was isolated from purified NEE using the ExoRNeasy Serum/Plasma Kit (Cat. No. 77023, Qiagen, Hilden, Germany) at Qiagen Genomic Services, Frederick, Md., USA. Briefly, 5 μl total RNA was used to prepare the miRNA NGS libraries using the QIAseq miRNA Library Kit (Cat. No. 331505, Qiagen, Hilden, Germany). RNA was ligated using adapters containing unique molecular indices (UMIs), and the RNA was converted to cDNA. Amplification of the cDNA was conducted using PCR (22 cycles) during which time PCR indices were added. Following purification, library preparation QC was conducted using a Bioanalyzer 2100 (Agilent Technologies Inc., Santa Clara, Calif., USA). Libraries were pooled in equimolar ratios based on quality of the inserts and the concentration measurements, quantified using qPCR, and sequenced on the NextSeq® 500 System (Illumina Inc., San Diego, Calif., USA). FASTQ files were prepared and checked following de-multiplexing of raw data (bcl2fastq software, Illumina Inc., San Diego, Calif., USA; FastQC). For trimming, adapter and UMI information from raw reads was extracted using Cutadapt v. 1.11 (Martin M. 2011 Cutadapt removes adapter sequences from high-throughput sequencing reads. EMBnet.journal 17, 10). Adapter sequences were removed and reads collapsed by UMI using a Qiagen in-house script. Reads were mapped using Bowtie2 v. 2.2.2 (Langmead B, Salzberg S L. 2012 Fast gapped-read alignment with Bowtie 2. Nat. Methods 9, 357-359) where the criteria for aligning reads to spike-ins, abundant sequence and miRbase (v20) specified that the reads perfectly match reference sequences. EdgeR (Robinson M D, McCarthy D J, Smyth G K. 2009 edgeR: a bioconductor package for differential expression analysis of digital gene expression data. Bioinformatics 26, 139-140) was used to calculate differential expression and data normalized using trimmed mean of M-values (TMM) normalization (Robinson M D, Oshlack A. 2010 A scaling normalization method for differential expression analysis of RNA-seq data. Genome Biol. 11, R25). miRNA was identified by mapping to miRBase (v20) (Kozomara A, Griffiths-Jones S. 2014 miRBase: annotating high confidence microRNAs using deep sequencing data. Nucleic Acids Res. 42, D68-D73). The reliability of the identified miRNAs is noted to increase with the number of identified fragments expressed in tags per million (TPM) (Robinson M D, et.al. 2009).

RNA Extraction for qPCR Quantitation of miRNA

Total RNA was extracted from the samples using ExoRNeasy Serum/Plasma Kit (Cat. No. 77023, Qiagen, Hilden, Germany) high-throughput bead-based protocol using the QIAcube Connect (Cat. No. 9002840, Qiagen, Hilden, Germany) at Qiagen Genomic Services. For miRNA quantitation, RNA was reverse transcribed to cDNA using the miRCURY locked nucleic acid (LNA) RT Kit (Cat. No. 339340, Qiagen, Hilden, Germany). The RNA Spike-In Kit for RT (Cat. No. 339390, Qiagen, Hilden, Germany) was applied to measure extraction efficiency and as quality control for RNA isolation and cDNA synthesis. Isolation controls were UniSp100 and UniSp101 in experiment 1 and UniSp2 and UniSp4 in experiment 2, and the cDNA synthesis controls for both experiments were UniSp3 and UniSp6. cDNA was diluted 50× and assayed in 10 μl qPCR reactions using the miRCURY LNA SYBR Green PCR kit (Cat. No. 339345, Qiagen, Hilden, Germany; Qiagen Genomic Services, Frederick, Md., USA); each miRNA sequence (hsa, Homo Sapien) was assayed once by qPCR for miR-23a-3p, miR-30c-5p, miR-103a-3p, miR-191-5p, and miR-451a for experiment 1 and miR-103a-3p, miR-23a-3p, miR-30c-5p, miR-142-3p and miR-451a for experiment 2. miR-103a-3p, miR-23a-3p, and miR-30c-5p are known to be expressed in a majority of sample types at a consistent concentration, and therefore were used to evaluate miRNA content of samples. To assess any contribution to miRNA signal from hemolysis in plasma samples, the ratio of the differential expression of miRNA-451a (highly expressed in thrombocytes) with miRNA-23a-3p (which has relatively stable expression in serum and is not affected by hemolysis), was determined. A ratio greater than 7.0 indicates an increased risk of hemolysis. Negative controls excluding template from the reverse transcription reaction was performed and profiled in the same manner as the samples and spike-ins.

cDNA qPCR

qPCR of cDNA generated from miRNA was conducted in accordance with MIQE guidelines (Bustin S A et al. 2009 The MIQE guidelines: minimum information for publication of quantitative real-time PCR experiments. Clin. Chem. 55, 611-622) at Qiagen Genomic Services. The relative quantitation of 34 target miRNAs selected from the NGS results was determined by qPCR using SYBR Green detection on a LightCycler® 480 Real-Time PCR System (Roche, Basel, Switzerland) in 384 well plates. A positive reaction is detected by accumulation of a fluorescent signal. The cycle threshold (Ct) is defined as the number of cycles required for the fluorescent signal to cross the threshold (i.e. exceed background amounts). The amplification curves were analysed using Qiagen software (v. 1.5.1.62 SP3), both for determination of Ct and for specificity, according to melt curve analysis.

### Data Analysis

The most stably expressed genes were selected as housekeeping genes by NormFinder (Andersen C L, Jensen J L, ØOrntoft TF. 2004 Normalization of real-time quantitative reverse transcription-PCR data: a model-based variance estimation approach to identify genes suited for normalization, applied to bladder and colon cancer data sets. Cancer Res. 64, 5245-5250.) and a geometric mean was calculated using the top 3 (miR-29b-3p, miR-126-5p and miR-146a-5p). Consideration to use additional house-keeping genes was evaluated following a stepwise inclusion protocol comparing the pairwise variation (Vn/n+1) calculated between two sequential normalization factors (Vandesompele J, De Preter K, Pattyn F, Poppe B, Van Roy N, De Paepe A, Speleman F. 2002 Accurate normalization of real-time quantitative RT-PCR data by geometric averaging of multiple internal control genes. Genome Biol. 3, RESEARCH0034). Since the pairwise variation of V¾ was below 0.05, we did not include a fourth house-keeping gene. All cycle-time expression values were, therefore, normalized to the geometric mean of the three most stable genes and standard equations were used to calculate delta Ct, delta-delta Ct and 2^{-(ΔΔCCt)}.

### Statistical Analysis

Differential expression analysis of the miRNA identified through NGSwas performed using EdgeR (Robinson et al. 2009) at QiagenGenomic services. For normalization, the trimmed mean of M-values method based on log-fold and absolute gene-wise changes in expression amounts between samples (TMM normalization) was used. Differential expression analysis was estimated by an exact test assuming a negative binomial distribution step (p<0.05 was determined as statistically significant).

We compared the gene fold expression [2−(ΔΔCt)] median scores for ALS/MND patients and controls in each of the two replicated experiments. Plots of the data distributions did not conform to normal distributions, so we used nonparametric analysis, specifically a two-tailed Mann-Whitney U Test, to examine two alternative hypotheses for each of the 34 miRNA sequences of interest:
H₀: the miRNA sequences were drawn from the same population, e.g. median values of 2^{-(ΔΔCt)} of the miRNA sequence are the same for ALS/MND patients and controls;
H₁: The miRNA sequences were drawn from different populations, e.g. median values of 2^{-(ΔΔCt)} of the miRNA sequence are different for ALS/MND patients and controls; with the null hypothesisH0 being rejected at p<0.05.

### Results

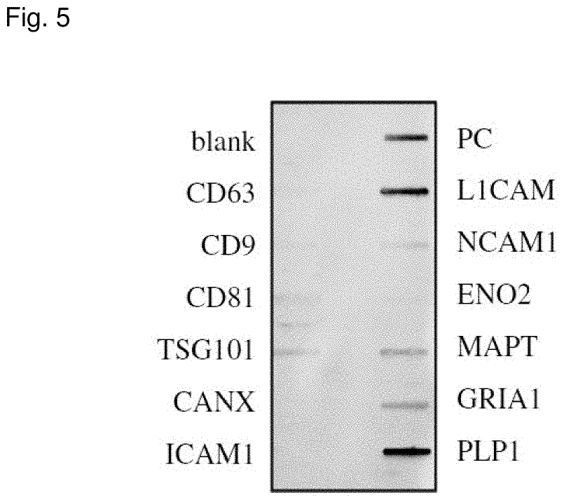

In two separate experiments using a different cohort of patients and controls for each experiment, we found eight miRNA sequences (Table 6) that were significantly and consistently different between ALS/MND patients and healthy controls (Table 5). Five miRNA sequences were upregulated in ALS/MND patients and three were downregulated (FIG. 4). The value and interpretation of these results is linked to the implementation of careful experimental quality controls which are reported below.

TABLE 5
Table 5. Differentially expressed miRNA as determined by qPCR of plasma samples comparing 10 ALS
patients and 10 controls reported from two identical, independent experiments (designated 1 and
2) using a different cohort of individuals in each experiment. Statistics were performed using
a two-tailed Mann-Whitney U-test. Median values refer to fold gene expression 2-(ΔΔCt). Fold
regulation was reported in a biologically relevant way and is defined as, fold change in cases
where fold change is greater than one, and in cases where fold change is less than one, fold regulation
equals negative one divided by fold change. Direction of fold regulation indicates differential
expression between ALS patients compared to healthy controls where an upregulation indicates higher
median expression in ALS patients and downregulation indicates decreased expression.

| | | | | median | median | fold | |
|---|---|---|---|---|---|---|---|
| experiment | miRNA ID | significance | Z-statistic | control | ALS | regulation | direction |
| 1 | miR-146a-5p | p < 0.05 | −2.02 | 1.00 | 1.21 | 1.2 | upregulated |
| 2 | miR-146a-5p | p < 0.05 | −2.44 | 1.03 | 1.43 | 1.4 | upregulated |
| 1 | miR-199a-3p | p < 0.05 | −2.44 | 0.97 | 1.28 | 1.4 | upregulated |
| 2 | miR-199a-3p | p < 0.001 | −3.38 | 1.08 | 2.86 | 2.7 | upregulated |
| 1 | miR-4454 | p < 0.05 | 2.44 | 1.10 | 0.54 | −1.7 | downregulated |
| 2 | miR-4454 | p < 0.05 | 2.4a4 | 0.97 | 0.44 | −1.8 | downregulated |
| 1 | miR-10b-5p | p < 0.01 | 2.61 | 1.00 | 0.58 | −2.1 | downregulated |
| 2 | miR-10b-5p | p < 0.0001 | 4.09 | 1.27 | 0.19 | −7.0 | downregulated |
| 1 | miR-29b-3p | p < 0.001 | 0.63 | 1.00 | 0.53 | −1.7 | downregulated |
| 2 | miR-29b-3p | p < 0.01 | 3.27 | 0.98 | 0.61 | −1.7 | downregulated |
| 1 | miR-151a-3p | p < 0.01 | −2.88 | 0.97 | 1.49 | 1.5 | upregulated |
| 2 | miR-151a-3p | p < 0.01 | −2.98 | 1.22 | 2.62 | 2.2 | upregulated |
| 1 | miR-151a-5p | p < 0.001 | −3.83 | 1.07 | 1.38 | 1.4 | upregulated |
| 2 | miR-151a-5p | p < 0.001 | −3.59 | 1.04 | 3.92 | 3.2 | upregulated |
| 1 | miR-199a-5p | p < 0.001 | −3.49 | 1.14 | 1.91 | 1.9 | upregulated |
| 2 | miR-199a-5p | p < 0.001 | −3.59 | 1.10 | 5.16 | 4.2 | upregulated |

TABLE 6
miRNA Sequences

| | SEQ | Mirabase | |
|---|---|---|---|
| mi-RNA | ID | (www.mirabase.org) | |
| name | NO: | Accession No. | Sequence |
| miR-146a-5p | 1 | MI0000477 | TGAGAACTGAATT |
| | | | CCATGGGTT |
| miR-199a-3p | 2 | MI0000242 | ACAGTAGTCTGCA |
| | | | CATTGGTTA |
| miR-4454 | 3 | MI0016800 | GGATCCGAGTCAC |
| | | | GGCACCA |
| miR-10b-5p | 4 | MI0000267 | TACCCTGTAGAAC |
| | | | CGAATTTGTG |
| miR-29b-3p | 5 | MI0000105 | TAGCACCATTTGA |
| | | | AATCAGTGTT |
| miR-151a-3p | 6 | MI0000809 | CTAGACTGAAGCT |
| | | | CCTTGAGG |
| miR-151a-5p | 7 | MI0000809 | TCGAGGAGCTCAC |
| | | | AGTCTAGT |
| miR-199a-5p | 8 | MI0000809 | CCCAGTGTTCAGA |
| | | | CTACCTGTTC |

**Any or all “T” nucleotide bases in the sequences of the miRNAs shown in Table 6 above may be substituted with a “U” nucleotide base.

### EV Characterization

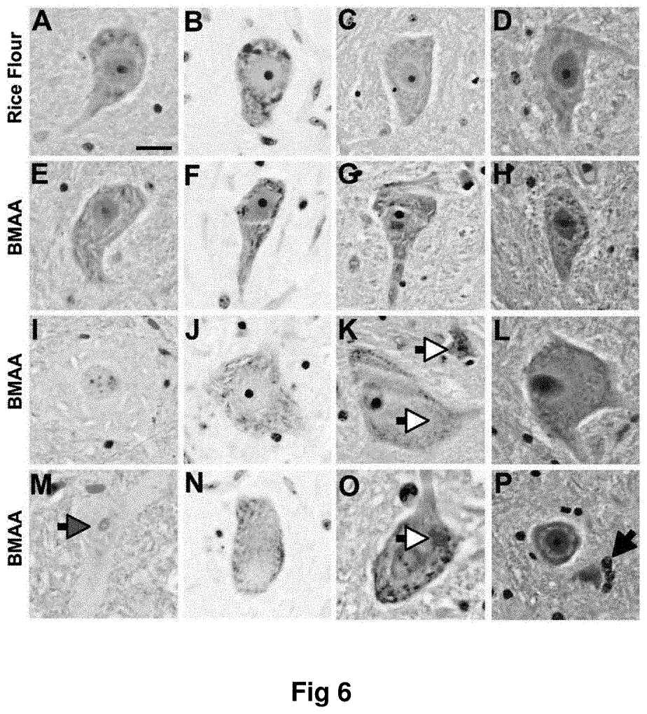

The absolute purity of the NEE fraction used in this experiment was not known, therefore, we refer to our extraction in the larger sense of extracellular vesicles sensu Théry et al. (Théry C et al. (2018) J. Extracell. Vesicles 7). Nevertheless, all indicators suggest that the miRNA expression reflect differences found within neural-derived exosomes. The particles recovered from our extraction procedures were of an appropriate size and composition to be consistent with exosomes (Théry et al. (2018); Witwer K W et al. (2013) J. Extracell. Vesicles 2; Hill A F, Pegtel D M, Lambertz U, Leonardi T, O'Driscoll L, Pluchino S, Ter-Ovanesyan D, Nolte-'t Hoen ENM. (2013) J. Extracell.Vesicles 2, 22859) with a median peak size of 102 nm for NEE (Table 7). Characterization of extracellular vesicles using nanoparticle tracking analysis suggest that vesicles are intact and within the parameters suggested as representative for exosomes (Table 7). The tetraspanins CD81 and CD63 which are known to be enriched in many exosomes were abundant and concentrated in NEE (CD81: 5.2-8.7, billion μl-1, n=20; CD63: 7.4-13.7 billion μl-1, n=6). We also note that tumour susceptibility gene 101 (TSG101), a component of the endosomal sorting complexes required for transport (ESCRT-I) complex, which is common in exosomes, was present and that a marker of cell contamination, calnexin, was absent. In the NEE fraction, we also found the presence of neural markers including: L1 transmembrane, neural cell adhesion, total tau, glutamate receptor 1 and proteolipid proteins (FIG. 5).

TABLE 7

| | neural-enriched EV | | total EV | |
|---|---|---|---|---|
| | fluorescence | scatter | fluorescence | scatter |
| | n = 3 | n = 3 | n = 4 | n = 4 |
| median peak size | 102 nm | 126 nm | 142 nm | 130 nm |
| full-width half-max | 95 nm | 98 nm | 101 nm | 106 nm |
| representation | 85% | 100% | 94% | 97% |
| span (90×-10×)/50× | 1.6 | 1 | 1.1 | 1.2 |

### Next Generation Sequence Pilot Study

The NGS pilot analysis was conducted to examine the quality and quantity of RNA within the NEE fraction, and to determine if the isolated NEE fraction differed from the T-N EV fraction in miRNA content. We successfully prepared, quantified and sequenced miRNA NGS libraries for all samples. The data passed all QC metrics; the NGS data had a high Q-Score (greater than 30), indicating good technical performance of the NGS experiment. An average of 4.6 million unique molecular index (UMI)-corrected reads per sample were obtained and the average percentage of mappable reads was 32.0%. We identified 256 miRNAs with ≥1 tags-per million mapped reads (TPM) and 149 with ≥10 TPMs. Results comparing UMI corrected reads (3.8 million: 5.3 million), miRNA/small RNA (6.6%:4.7%) and mapped genome (23.9%:23.8%) did not reveal notable differences between the two groups T-N and NEE, respectively (n=4 for each category). NormFinder analysis returned 25 stably expressed miRNAs, including the constitutively expressed miR-103a-3p with abundance measures between 294 and 3966 average TPM. Comparison between T-N and NEE identified 39 differentially expressed miRNA (p-values<0.05), within this small NGS pilot study.

### NGS Analysis of 10 ALS/MND Patient and 10 Control Plasma Using NEE

Next generation sequencing analysis identified miRNA, small RNA, genome-mapped, out-mapped, high-abundance RNA and unmapped reads, the latter of which did not align to the genome. We characterized miRNA as having 18-23 nucleotide lengths. The average number of UMI-corrected reads per sample was 6.6 million, with an average percentage of mappable reads of 61.5%, indicating usable data.

Expression Levels of miRNA

A total of 350 miRNAs were identified with a call rate ≥1 TPM and 219 were found to have a call rate ≥10 TPM. Statistical analysis of NGS data from extracellular vesicles (NEE) extracted from the plasma of 10 healthy control and 10 ALS/MND patients returned 101 significantly differentially expressed miRNA (p<0.05). From these 101 miRNA, 34 were chosen for relative-quantification using qPCR.

qPCR Quantitation; miRNA QC Results

We observed a steady amount of expression of UniSp3 and UniSp6 in every sample indicating the RT and qPCR reactions were successful. Similar Cts for all negative controls indicate none of the samples contained inhibitors. One sample had a slightly elevated hemolysis ratio (greater than 7.0), which we determined to be insufficient to exclude from further analysis. We report Cts between 20 and 30 within all 40 samples, indicating there was enough miRNA to proceed with the downstream experiments.

qPCR Quantitation of Differentially Expressed miRNA

From the 101 species of miRNA identified as differentially expressed by NGS, we selected 34 for downstream qPCR quantitation. The criteria for selection was based on, (1) miRNA that were significantly differentially expressed between the controls and the patients, as determined by NGS, and; (2) miRNA that were detected in the NGS and had previously been identified in the literature as being of interest in motor neuron disease.

Statistical analysis of the 34 target miRNA sequences from n=10 controls and n=10 patients returned highly significant comparative data, suggesting robust results. Thus, we repeated the entire experiment using identical methods but using a new cohort of patient and control samples. Statistical analysis of the replicate experiments returned eight miRNA that were differentially expressed between NEE derived from ALS/MND patients in comparison with a healthy control population of equal size in both experiment one and two (table 7). The following miRNA were analyzed by qPCR but were found to be not significantly different between ALS/MND patients and controls, or not significantly different in one of the two experiments conducted using different patient cohorts which makes them not sensitive enough for use as ALS/MND neural-derived exosomes: let-7b-5p, let-7d-3p, let-7d-5p, miR-126-3p, miR-126-5p, miR-133a-3p, miR-1-3p, miR-143-3p, miR-146a-3p, miR-194-3p, miR-23a-3p, miR-330-3p, miR-338-3p, miR-339-3p, miR-339-5p, miR-451a, miR-517a-3p, miR-584-5p, miR-625-3p, miR-708-5p and miR-744-5p.

### Discussion

We identified eight miRNA sequences derived from NEE extractions that consistently and significantly differentiate ALS/MND patients from healthy controls with a single blood draw. These miRNA sequences were drawn from two experiments using different patient and control cohorts each producing the same eight miRNA sequences. We suggest that these miRNA sequences, singly or in combinations, can confirm the diagnosis of ALS/MND based on standard clinical criteria and may allow ALS/MND to be diagnosed in pre-symptomatic individuals, rapidly speeding diagnosis and treatment. Furthermore, the upregulation or downregulation of these miRNA sequences may potentially allow the effectiveness of existing or novel treatments of ALS/MND to be rapidly assessed before any clinical changes in patient disease progression or symptoms.

EVs drawn from blood plasma are important reservoirs for neural-derived exosomes for three reasons: (1) they are stable and abundant in biological fluids; (2) blood plasma is routinely drawn from patients and this procedure is relatively non-invasive when compared with lumbar punctures or tissue biopsies; and (3) the cargo of EVs contain important biomolecules including nucleic acids and proteins. Owing to unique proteins on the surface of EVs, subpopulations of specific origins can be enriched. For example, the presence of L1CAM was used in our experiments to isolate a sub-population of neural-enriched EVs. Although L1CAM is not exclusively expressed in the brain, the enrichment process enhances the chance that the neural-derived exosomes found are related specifically to neurodegeneration. The consistency of the results across two independent experiments achieved in this report supports this method as being robust and useful in the discovery of neural-derived exosomes. We examine only miRNA in this study, but it may be possible to use the same extraction and enrichment techniques to examine proteins, lipids, and other RNA species for neural-derived exosome potential.

Katsu et al. (Katsu M, Hama Y, Utsumi J, Takashina K, Yasumatsu H, Mori F, Wakabayashi K, Shoji M, Sasaki H. (2019) Neurosci. Lett. 708, 134176), in a study with five ALS patients and five control patients using extraction methods similar to ours, identified 30 miRNA that differed between the two groups but these were not verified by qPCR. This contrasts with our study that had a larger sample size (20 per independent experiment×2 experiments=40 total individual samples) and was validated by qPCR. Data from our NGS experiment, which helped to identify miRNA sequences of interest for the more rigorous qPCR analysis, revealed only two overlapping miRNA sequences (miR-24-3p, miR-150-3p) with Katsu et al. neither of which we chose for further study. Katsu et al. suggested that the data they presented should be validated by qPCR and that larger patient cohorts are needed to determine the broader application of the identified miRNA to ALS. The majority of the miRNA found by Katsu et al. were not identified in our NGS study indicating that they were either not present in the samples we examined, or that their abundance was sufficiently low as to not be recognized. Of the two miRNA that did overlap between our NGS study and the Katsu et al. study, we evaluated both after the necessary statistical adjustment for false discovery rates (FDR) and found that miR-24-3p was not significant between ALS/MND patients and controls (FDR p-value=0.053), but that miR-150-3p was significant between ALS/MND patients and controls (FDR p-value=0.006). These two miRNA that we identified in NGS have not undergone qPCR evaluation, therefore we cannot determine the importance of these miRNA as possible ALS neural-derived exosomes. We chose not to report the other possible 67 identified miRNA from our NGS analysis until they can be further analysed in a more quantitative fashion.

Some miRNAs have been considered as potentially valuable for ALS/MND patient neural-derived exosome investigation by other researchers using CSF, peripheral blood leucocytes, muscle tissue or plasma/serum in the absence of extracellular vesicle isolation. It is difficult to parse the comparable value of miRNA from different biological fluids and different methods of extraction and analysis. We do note that of those miRNA identified in other studies did not meet the criteria to reject a hypothesis of similar expression values between ALS/MND patients and healthy controls in our sample population: let-7b-5p, let-7d-3p, let-7d-5p, miR-126-5p, miR-133a-3p, miR-143-3p, miR-146a-3p, miR-23a-3p, miR-338-3p, miR-451a, miR-584a-5p. miR-146a-5p, miR-151a-5p, miR-199a-3p, miR-199a-5p were consistently significant in our analysis. In our study, miR-146a-5p was analyzed by qPCR and shown to be upregulated in NEE of two separate experiments using a different cohort of patients. We suggest that the extraction protocol followed in this study, extracting extracellular vesicles from blood plasma followed by enrichment of neural vesicles using L1CAM, leads to a more relevant pool of miRNA that is directly associated with motor neuron processes, is repeatable, and is useful as neural-derived exosomes for ALS/MND. The replication reported here using different cohorts of patients and controls supports this assertion.

miR-146a-5p is known to be involved in both influencing synaptic plasticity [61] and regulating the inflammatory response. We report that miR-146a-5p was upregulated in the NEE of ALS/MND patient samples versus controls. The precise function of miR-146a-5p in ALS, however, could also be related to a role in anti-inflammation particularly within astrocytes. We note that 146a has been found to be upregulated in AD brain tissues and downregulated in plasma, serum, and CSF of Alzheimer's patients. 146a was not found to be upregulated in four ALS, four PD, or five schizophrenia temporal lobe neocortex tissues when compared with six control tissues.

### Conclusion

We successfully extracted, enriched and characterized a neural-derived subpopulation of EVs from multiple ALS/MND patient and control samples and determined that they contain sufficient miRNA to conduct NGS and qPCR. In repeated experiments using different patient and control cohorts, we have identified eight miRNA sequences that are differentially expressed in ALS/MND patients and healthy controls. This replication provides strong evidence that these miRNA sequences individually or in combination should be further investigated as ALS/MND neural-derived exosomes using larger sample sizes. Further work to compare these results with other motor neuron conditions is also warranted.

As provided herein, it has been demonstrated that miRNA sequences derived from neural-enriched exosome fractions may be used as neural-derived exosomes to distinguish, for example, ALS patients from baseline and/or from control subjects in a highly replicable manner. Accordingly, miRNA sequences, alone, or in combination, including one or more of miR-146a-5p, miR-199a-3p, miR-4454, miR-10b-5p, miR-29b-3p, miR-151a-3p, miR-151a-5p, and miR-199a-5p, may be used as a motor neuron disease neural-derived exosome, such as an ALS neural-derived exosome.

This invention will allow ALS to be diagnosed from a single blood draw. While the ALS samples were drawn from a pool of patients with diagnosed ALS, these miRNA sequences, singly or in combination, will allow ALS to be diagnosed in pre-symptomatic individuals, rapidly speeding diagnosis and onset of treatment. Furthermore, the upregulation or down regulation of these miRNA sequences will allow the potential effectiveness of existing or novel treatments of ALS to be rapidly assessed before any changes in patient disease progression or symptoms. Therefore, analysis of these miRNAs, singly or in combination, represent a new method for rapidly screening new treatments for efficacy in ALS, as well as other progressive motor neuron diseases.

Neural-derived exosomes for amyotrophic lateral sclerosis/motor neuron disease (ALS/MND) are currently not clinically available for disease diagnosis or analysis of disease progression. If identified, neural-derived exosomes could improve patient outcomes by enabling early intervention and assist in the determination of treatment efficacy. We hypothesized that neural-enriched extracellular vesicles could provide microRNA (miRNA) fingerprints with unequivocal signatures of neurodegeneration. Using blood plasma from ALS/MND patients and controls, we extracted neural-enriched extracellular vesicle fractions and conducted next-generation sequencing and qPCR of miRNA components of the transcriptome. We here report eight miRNA sequences which significantly distinguish ALS/MND patients from controls in a replicated experiment using a second cohort of patients and controls. miRNA sequences from patient blood samples using neural-enriched extracellular vesicles may yield unique insights into mechanisms of neurodegeneration and assist in early diagnosis of ALS/MND.

### Example 4—L-Serine Reduces Spinal Cord Pathology in a Vervet Model of Preclinical ALS/MND

The early neuropathological features of amyotrophic lateral sclerosis/motor neuron disease (ALS/MND) are protein aggregates in motor neurons and microglial activation. Similar pathology characterizes Guamanian ALS/Parkinsonism dementia complex (PDC), which may be triggered by the cyanotoxin β-N-methylamino-L-alanine (BMAA). We report here the occurrence of ALS/MND-type pathological changes in vervet primates (Chlorocebus sabaeus; n=8) fed oral doses of a dry powder of BMAA HCl salt (210 mg/kg/day) for 140 days. Spinal cords and brains from toxin-exposed vervets were compared to controls fed rice flour (210 mg/kg/day) and to vervets coadministered equal amounts of BMAA and L-serine (210 mg/kg/day). Immunohistochemistry and quantitative image analysis were used to examine markers of ALS/MND and glial activation. UHPLC-MS/MS was used to confirm BMAA exposures in dosed primates. Motor neuron degeneration was demonstrated in BMAA-dosed vervets by TDP-43+ proteinopathy in anterior horn cells, by reactive astrogliosis, by activated microglia, and by damage to myelinated axons in the lateral corticospinal tracts. Vervets dosed with BMAA+L-serine displayed reduced neuropathological changes. This study demonstrates that chronic dietary exposure to BMAA causes ALS/MND-type pathological changes in the vervet and coadministration of L-serine reduces the amount of reactive gliosis and the number of protein inclusions in motor neurons.

### Materials and Methods

### Toxin Dosing

Young adult male vervet primates (Chlorocebus sabaeus; age 7 years; 3.1 kg) were housed in groups inside large outdoor enclosures at the Behavioural Science Foundation (B SF) (St. Kitts, West Indies). The BSF is a fully accredited biomedical research facility with approvals from the Canadian Council on Animal Care. The BSF Institutional Animal Care and Use Committee approved the use of our experimental protocol for this study. During the dosing experiment, vervets ate a low-protein diet comprising predominantly local fruits and vegetables. For dietary exposure, L-BMAA HCl, L-BMAA HCl plus L-serine, or rice flour was placed inside a cavity of a banana and presented to vervets prior to their daily allotment of food. L-BMAA HCl salt was synthesized by Irvine Chemistry Lab (Anaheim, Calif.), with purity confirmed by ¹H NMR and ¹³C NMR. Optical rotation was c=1.15 mg/mL in 5 N HCl, 27.7° C., with a melting point of 186-190° C. (Cox et al 2016 Supplementary Material). In tandem LC/MS/MS, the synthesized BMAA was consistent in mass, product ions, and product ion ratios with an authenticated standard (B-107, Sigma-Aldrich, St. Louis, Mo.). Vervets were randomly assigned to one of three 8-member cohorts for 140 days of dosing with L-BMAA HCl at 210 mg/kg/day, L-BMAA HCl plus L-serine both at 210 mg/kg/day, and a control cohort dosed with 210 mg/kg/day of rice flour. The 140-day BMAA dosing regimen of the adult vervet was calculated to be equivalent to the 20-year lifetime exposure of an adult Chamorro male consuming a 30-g serving of cycad powder per day in tortillas and 8 flying foxes (Pteropus mariannus) per month. Group enclosures for each vervet cohort were spaced appropriately apart to reduce the chance for sharing bananas to cause cross contamination. The 210 mg/kg of powdered L-BMAA HCl de-livered an effective daily dose of 161 mg/kg L-BMAA HCl. The doses were prepared at Brain Chemistry Labs (Jackson, Wyo.) using a Mettler Toledo balance with a Quantos automated powder-dispensing module at a tolerance of 60.1% of target dose. To ensure oral delivery of test sub-stances, veterinarian, BSF and Brain Chemistry Labs staff monitored daily dosing. Cerebral spinal fluid, hair, and plasma were sampled every 4 weeks from each vervet under ketamine anesthesia, and body weight was recorded. All vervets were observed daily for mortality, morbidity, and clinical signs of adverse health effects and qualitative food consumption.

### Necropsy and Histopathology

After the 140-day, chronic dosing regimens, vervets were euthanized under ketamine anesthesia followed by complete dissection of brain and spinal cord tissues. All the external surfaces of specimens were examined, photographed, and fixed in 10% neutral buffered formalin for histopathological studies. Brain tissues were placed into phosphate buffered saline (PBS), pH 7.4, and shipped to NeuroScience Associates (Knoxville, Tenn.) for processing, embedding, and sectioning into 40-μm sections on 76×51mm glass slides using the MultiBrain Service. Spinal cords were processed using xylenes and alcohols and coronal segments were embedded in paraffin wax in preparation for Leica microtome sectioning into 7-μm sections on 75×25 mm glass slides. Slide mounts of 3 cervical and 3 lumbar spinal cord segments were deparaffinized in 3 changes of xylene for 10 minutes each, followed by 2 changes of absolute ethanol for 5 minutes each, then 95% ethanol for 5 minutes. Routine histological stains included he-matoxylin and eosin (H&E), periodic acid-Schiff, Luxol fast blue, thionine-Nissl, and thioflavin-S. To probe for specific protein antigens after hydration, slide tissue mounts were incubated in 3% H₂O₂ in methanol for 10 minutes followed by rinsing in distilled water for 3 changes of 5 minutes. Slides were then incubated in antigen retrieval buffer (98% formic acid for 45 seconds or citrate buffer for 30 minutes, where applicable), followed by a wash in 3 changes of distilled H₂O on a Thermolyne Roto Mix shaker and incubation in PBS for 5 minutes. To block nonspecific antibody binding, 10% normal donkey serum (NDS) in PBS was applied to slides in a humidity chamber and incubated at room temperature for 30 minutes. Brain and spinal cord slide mounts were probed with antibodies against β-amyloid (A_{β}:1:800, Covance, Princeton, N.J.), cluster of differentiation 68 (CD68: 1:500, DAKO/Agilent, Calif.), fused in sarcoma (FUS:1:2000, Novus Biological, Centennial, Colo.), ionized calcium binding adaptor molecule 1 (IbA1:1:300, FUJIFILM Wako Chemicals, Richmond, Va.), glial fibrillary acidic protein (GFAP:1:1000, Sigma-Aldrich), phospho-tau (Ser202, Thr205) monoclonal antibody (ATB: 1:3000, Thermo Fischer Scientific, Waltham, Mass.), phosphorylated TAR DNA-binding protein 43 (TDP-43: 1:1000, Cosmo-Bio, Carlsbad, Calif.), and ubiquitin (Ubiq: 1:300, Millipore, Waltham, Mass.). Slides were incubated with antibodies at 4° C. overnight. Slides were rinsed with PBS for 3 changes of 10 minutes, followed by additional application of 2% NDS for 10 minutes prior to incubation with secondary antibodies. A donkey anti-mouse biotin (1:200; Jackson Immunoresearch, West Grove, Pa.) conjugated secondary antibody goat anti-mouse/or anti-rabbit was incubated on tissue sections for 2 hours at room temperature, rinsed with PBS wash for 10 minutes, and followed by application of ExtrAvidin peroxidase (1:5000, Sigma-Aldrich) in PBS for 1 hour. ExtrAvidin peroxidase was detected using diaminobenzidine (DAB) solution (100 mL DAB 98 mL PBS 2 mL 25 mg/mL DAB 16.6 μL 3% H₂O₂) for 10 minutes. Slides were washed in 2 changes of PBS, rinsed with distilled water, and counterstained with Gill No. 1 hematoxylin for 20 seconds and rinsed under running tap water for 5 minutes. Phospho-Tau AT8 immunohistochemistry was performed on cortical brain sections by NeuroScience Associates. Sevier Münger silver staining was performed at AML Laboratories (Jacksonville, Fla.) and counterstained with Gill No. 1 hematoxylin. Postmortem spinal cord and brain tissues from a 58-year-old Caucasian male with a neuropathologically confirmed diagnosis of sALS were used as positive controls with each immunostaining protocol.

### Digital Pathology and Microscopy

Histology slides were scanned at 40 resolution using a TissueScope LE (Huron Digital Pathology, Waterloo, Ontario, Canada). Digital scanning allowed for complete mapping of entire spinal cord sections and clear visualization of margins and anatomical landmarks at an optimal resolution of (0.2 lm/pixel [Px] at 40) for image analysis. High quality tiff image file (1721 985 Px or 3259 1174 Px) fields were exported from TissueScope LE and imported in NIH ImageJ 64 VER1.44o (NIH, Bethesda, Md.) for analysis. To determine the total area, number, and size of glial cells and neurons, 12 tiff images were analyzed per vervet (6 fields per cervical and 6 fields per lumbar spinal cord segments) (data not shown). Spinal cord tissue sections were blinded and scored manually and compared to automated counting. For automated analysis, an unbiased threshold was applied to regions of interest to determine the morphological findings across treatment groups (data not shown). Slides stained with thioflavine-S and 4′,6-diamidino-2-phenylindole (DAPI) were visualized at 20 magnification using a Zeiss Apotome fluorescent microscope (Thornwood, N.Y.).

### Detection and Quantification of BMAA Toxin

Spinal cord tissues were analyzed to measure the concentration of BMAA using triple quadrupole tandem mass spectrometry (UHPLC-MS/MS) with a precolumn 6-amino-quinolyl-N-hydroxysuccinimidyl carbamate (AQC) derivatization employing a method validated according to peer AOAC International guidelines and previously reported (Banack S A, et al., (2018) Neurotox Res 2018; 33:24-32; Glover W B, et al., (2015) J AOAC Int 98:1559-65) (data not shown). Spinal cord samples (50 mg) were extracted with TCA (20% w/v) for free BMAA followed by HCl (6.0 M) hydrolysis of the pellet at 110° C. for 16-18 hours to release protein-bound BMAA. The presence of BMAA in the supernatant after TCA extraction was checked by further HCl hydrolyzation of the supernatant. Extracted samples were filtered using a centrifuge filter (0.2 μm, Milli-pore UltrafreeMC) at 14,000 g for 5 minutes and analyzed on a Thermo Scientific TSQ Quantiva triple quadrupole mass spectrometer attached to a Thermo Vanquish Ultra-High Pressure Liquid Chromatography Autosampler equipped with a Vanquish pump and column compartment. Separation of 4 BMAA structural isomers (N-[2-aminoethyl] glycine, 2,3-diaminobutanoic acid, 2,4-diaminobutyric acid, and β-amino-N-methyl-alanine) was achieved with gradient elution using 20 mM ammonium acetate, pH 5.0 (A) and 100% methanol (B) as follows: 0.5 mL/minutes, 0 minutes 10% B, 1.0 minutes 10% B, 4.8 minutes 40% B (curve 5), 5.0 minutes 90% B (curve 5), 6.8 minutes 90% B, 6.81 minutes 10% B (curve 5), and 8 minutes 10% B. Separation was performed using a Thermo Hypersil Gold C-18 column (PN 25002-102130) 100×2.1 mm, particle size 1.9 μm heated to 65° C. Samples were analyzed in positive ion, single reaction monitoring mode using heated electrospray ionization using previously published ion transitions (Cox PA, et al., (2016) Proc Biol Sci 283:2015239). Mass spectrometer ion source properties were as follows: 3500 V-positive ion, 45 Arb Sheath gas, 10 Arb Aux gas, Sweep gas 0.1 Arb, vaporizer temperature 400° C., and ion transfer tube temperature 350° C. Validation curves and parameters were performed as in Glover et al (U.S. Food and Drug Administration, Center for Drug Evaluation and Research. Reviewer Guidance: Validation of Chromatographic Methods. Washington, D.C. 1994) passing all criteria exceeding minimum requirements for a single-laboratory validation. LOD (0.009 ng/mL) and LLOQ (0.037 ng/mL) were calculated according to FDA recommended regulatory guidelines (U.S. Food and Drug Administration, Center for Drug Evaluation and Research. Reviewer Guidance: Validation of Chromatographic Methods. Washington, D.C. 1994). All samples were run and normalized with an internal BMAA standard (β-N-methyl-d₃-amino-_{D}L-alanine-¹⁵N2) at a concentration of 1.5 ng/mL. System blanks (AQC derivatized blanks, internal standards, and deionized water) were injected between sample injections.

### Statistical Analyses

Prism Version 7 software (Graph Pad, La Jolla, Calif.) was used to perform statistical analyses. Single comparisons tests were analyzed using Student t test and Mann-Whitney U test. Multiple comparisons were analyzed using one-way analysis of variance (ANOVA) with Newman-Keuls Multiple Comparison test, the Friedman with Dunn's multiple comparison tests, or the Wilcoxon match pairs sign test. For correlation analyses, Pearson's correlation coefficient and Spearman correlation tests were used to determine significance. D'Agostino and Pearson test was used to determine normality of each data set. All data were expressed as the median ± standard error; significance level of α=0.05. Due to the limited size of our sample cohort, Cohen's f was used to determine effect size.

TABLE 8
Detection of Free and Protein-Bound BMAA in Vervet Spinal Cord

| | | Hydrolyzed Protein | Hydrolyzed |
|---|---|---|---|
| Exposure Cohort | Free (μg/g) | Pellet (μg/g) | Supernatant (μg/g) |
| Rice flour | ND | ND | ND |
| BMAA | 8.9 ± 3.9 ^{NS} | 0.5 ± 0.3 ^{NS} | 34.0 ± 20.5 ^{NS} |
| BMAA + L-serine | 9.4 ± 2.2 | 0.6 ± 0.1 | 56.2 ± 13.6 |

Median ± SEM of BMAA per gram of spinal cord tissues;
ND, not detected;
^{NS} no statistically significant differences were found between groups using a nonparametric Mann-Whitney U test.

### Results

### Toxin Accumulation

Spinal cord tissues collected at necropsy from BMAA and BMAA L-serine cohorts were positive for the cyanobac-terial toxin (Table 8 and data not shown). As expected, the rice flour control group had no detectable levels of the BMAA (Table 8). The median total BMAA concentration in spinal cord tissues was 61.4 μg/g±13.7 SE and ranged from 15.8 to 199.9 μg/g in the BMAA and BMAA L-serine dosing cohorts. The median and range of concentration of detect-able BMAA in vervets were similar to those measured in cortical brain tissues from individuals with Guam ALS/PDC and spinal cords from North Americans with ALS/MND. The concentrations of free and protein-bound BMAA were positively correlated (r 0.919, p<0.0001). In addition to these 2 protein fractions, considerable BMAA was found in the hydrolyzed supernatant indicating small soluble peptides present in spinal cord tissues (Table 8). Coadministration of L-serine did not significantly reduce the median concentrations BMAA, in any fraction, of the vervet spinal cord (Table 8 and data not shown).

### Anterior Horn

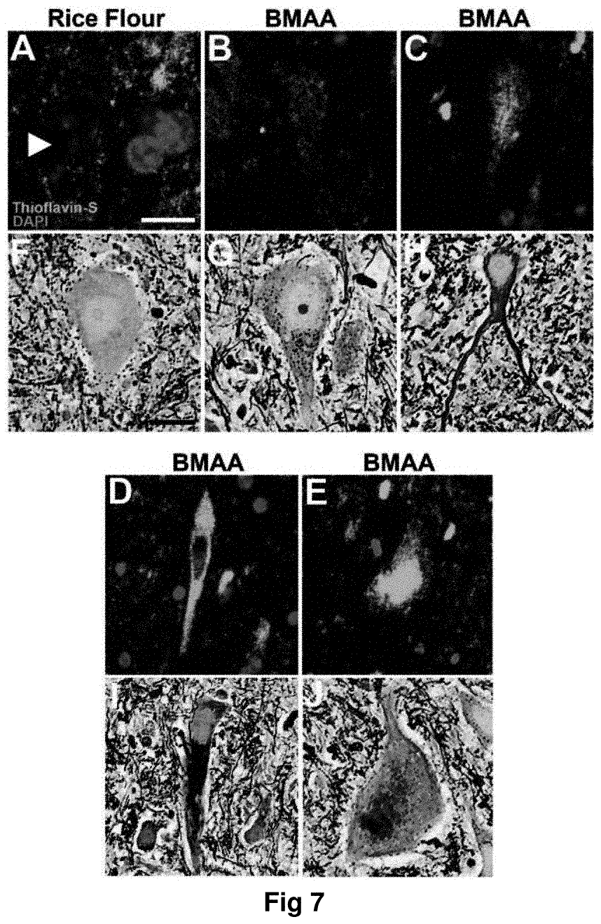

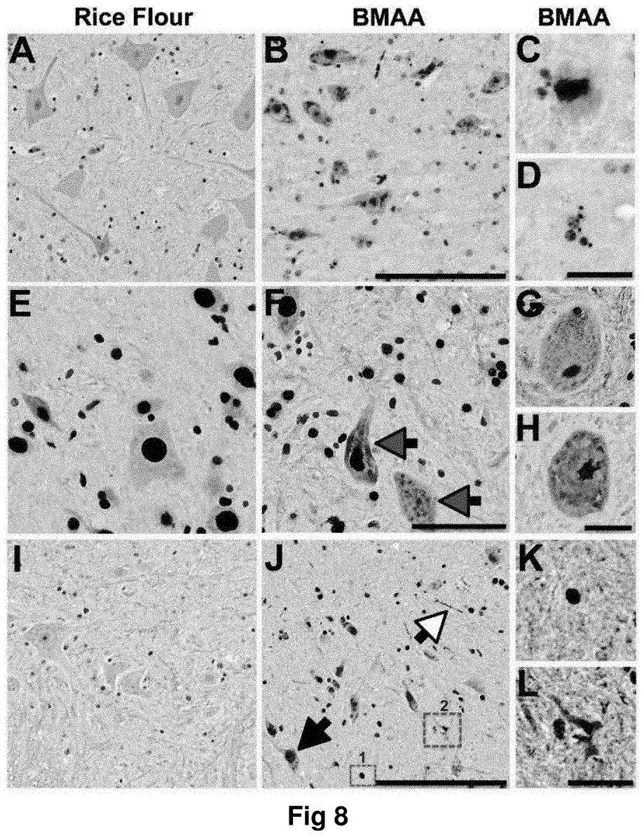

Microscopic examination of lower motor neurons of the cervical spinal cord anterior horns of BMAA-dosed vervets showed 4.3-fold greater frequency of eosinophilic neurons (p=0.024) that were reduced 40% in size (p<0.0001) and contained abundant skeinlike cytoplasmic vacuoles (p=0.0002) that were absent in controls dosed with rice flour (FIG. 6; A-H and data not shown). Anterior horn neuronal numbers were decreased by 23% (p=0.0016) and a portion was observed to have thinning of Nissl substances and chromatolysis (FIG. 6; I-L, N and data not shown). Bunina bodies, a pathological hallmark of ALS/MND (37) and Guam ALS/PDC (11), were also observed in spinal cord motor neurons of vervets dosed with BMAA (FIG. 6; M). The accumulation of glycogen (FIG. 6; K, O), a sign of metabolic dysfunction, along with cell death and neuronophagia was also present in anterior horn neurons (FIG. 6; P). Other qualitative markers of ALS/MND-type cellular injury included the intracellular accumulation of thioflavin-S⁺ inclusions and dystrophic argyrophilic neurons similar to NFTs were abundant in the BMAA-dosed cohort, but not observed in rice flour-fed primates (FIG. 7). Extracellular amyloid beta⁺ plaques were not observed in any cohort.

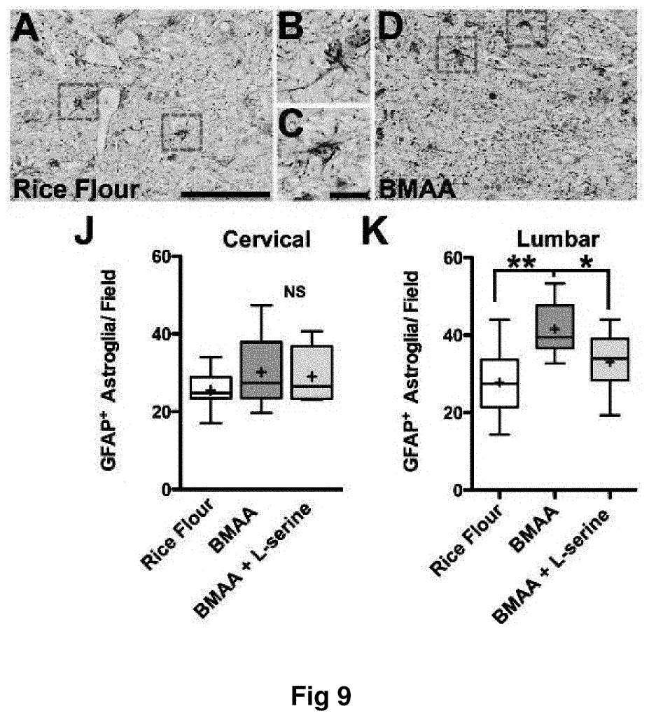

Protein inclusions in anterior horn cells typically associated with the diagnosis of ALS/MND were also found in the BMAA-dosed vervet cohorts. Neurons from 12 of 16 (75%) of vervets fed BMAA were positive for TDP-43 dense, granular, and round cytoplasmic inclusion bodies (FIG. 8; A-D and Table 9) (Mori F, et al., (2008) Acta Neuropathol 116:193-203). The mean density and distribution of TDP-43⁺ inclusions in toxin-dosed vervets were 5-fold greater (p=0.024) than those observed in controls, which is similar to that observed in Guam ALS/PDC (Table 9). Rare cytoplasmic mislocalization of FUS protein, a marker used in postmortem diagnosis of TDP-43-negative ALS/MND patients, was observed in 2 of the 16 (12.5%) BMAA-dosed vervets (FIG. 8; E-H). Anterior horn neurons and glia also displayed UBIQ⁺ cytoplasmic inclusions and neurites (FIG. 8; I-L). The presence of both TDP-43⁺ and UBIQ⁺ protein inclusions along with Bunina bodies in anterior horn neurons suggests that chronic BMAA dosing causes motor neuron injury in vervets characteristic of ALS/MND and Guam ALS/PDC.

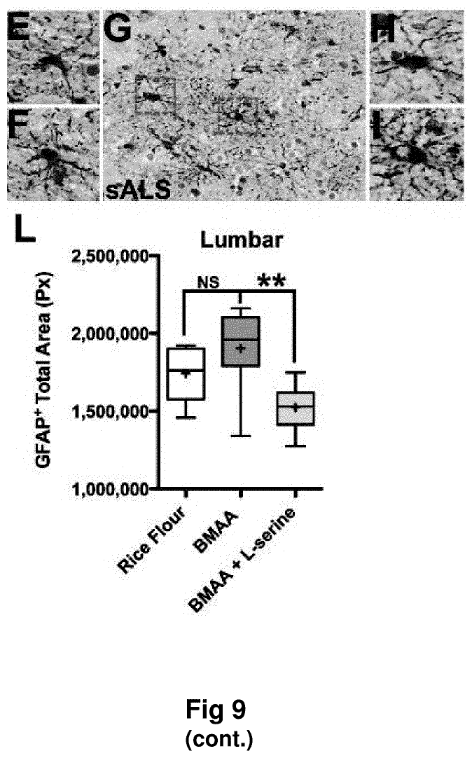

Astroglia are nonneuronal cells that support neuronal plasticity, modulate synaptic transmission and have been previously shown to be susceptible to BMAA toxicity. In our study, vervets receiving fruit supplemented with rice flour displayed only astroglia with normal cellular architecture that were distributed adjacent to healthy motor neurons in the spinal cord (FIG. 9; A-C). Chronic dietary dosing with BMAA increased the density of GFAP⁺ activated astroglia 1.4-fold (p=0.008) in the vicinity of atrophic and vacuolated motor neurons in the anterior horns of the lumbar spinal cord (FIG. 9; D-F, K). GFAP⁺ astroglia in BMAA-dosed vervets displayed morphological changes similar to those found in a sALS patient (FIG. 9; G-I). Reactive astrogliosis was also observed in the primary motor cortex and midbrain with decreasing density progressing down the neuroaxis of toxin-dosed vervets (data not shown). Coadministration of L-serine reduced the total number (p=0.008) and total area (p=0.007) of GFAP⁺ astroglia in the anterior horn of the lumbar spinal cord by 21% and 20%, respectively (FIG. 9; K, L).

### Lateral Corticospinal Tract

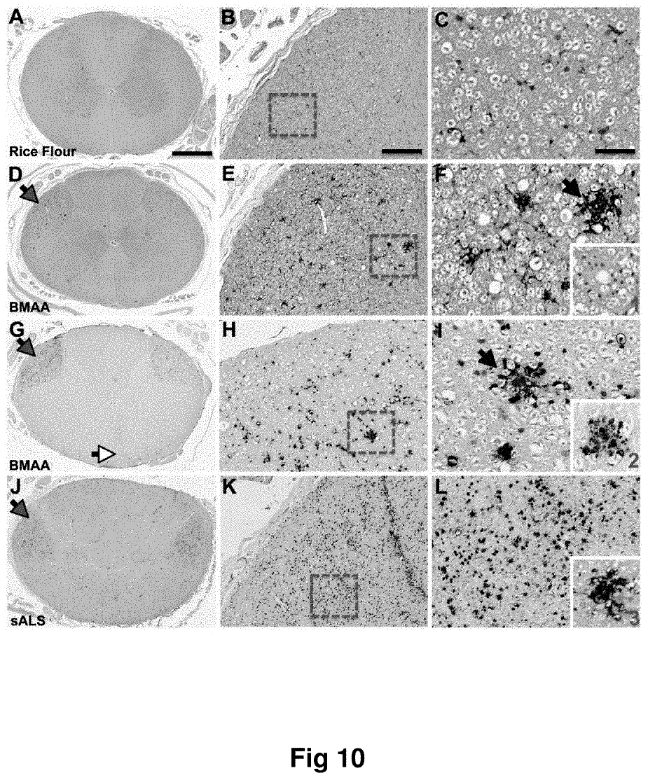

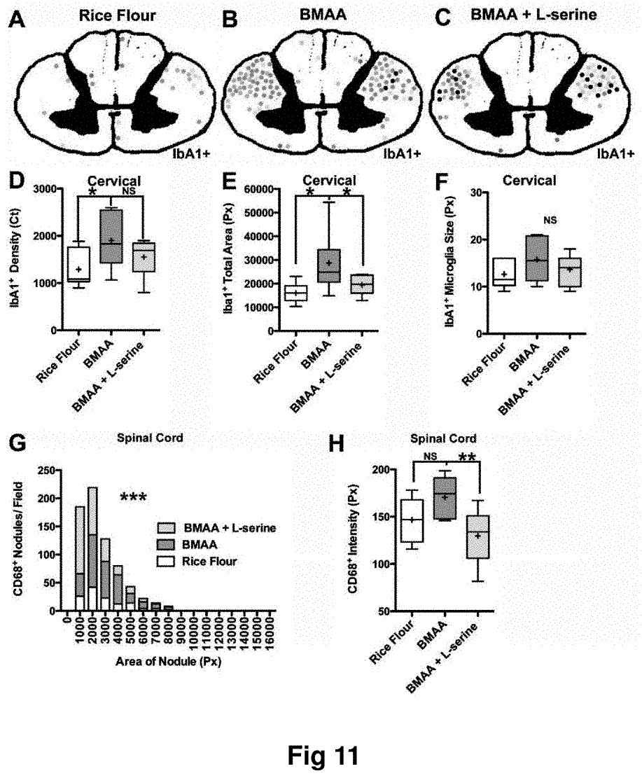

Microglia are resident macrophage/immunocells of the central nervous system that play an important role in synaptic regulation and modulation of neuron networks, as well as inflammation and scavenging. Microglial activation is one of the earliest pathological changes in ALS/MND (Geloso M C, et al., (2017) Front Aging Neurosci 9:242). Chronic dietary dosing with BMAA increased the density 1.7-fold (p=0.048) and total area 1.6-fold (p=0.011) of IbA1⁺ microglia in the cervical spinal cord of vervet primates compared to controls fed rice flour (FIG. 10; A-F and FIG. 11; A-F). BMAA effects on IbA1⁺ microglia in the spinal cord were region-specific and targeted predominantly to descending motor pathways. Ascending white matter tracts such as the dorsal column, the spinocerebellar, and the anterolateral system were unaffected (FIG. 10; D and FIG. 11; B, C and data not shown). IbA1⁺ microglia were observed forming large nodule-type lesions and phagocytes in the lateral corticospinal tracts of BMAA-dosed vervets, but not in rice flour controls (FIG. 10; A-F and FIG. 11; A-F). The density and the total area covered by IbA1⁺ microglia were 1.5-fold (p=0.0039) greater in the cervical compared to the lumbar segments of the spinal cord from BMAA-dosed primates (Table 10). Iba1⁺ microglia nodules were also observed in the pyramids of the medulla, the cerebral peduncles, and cortical areas of BMAA-dosed vervets (data not shown).

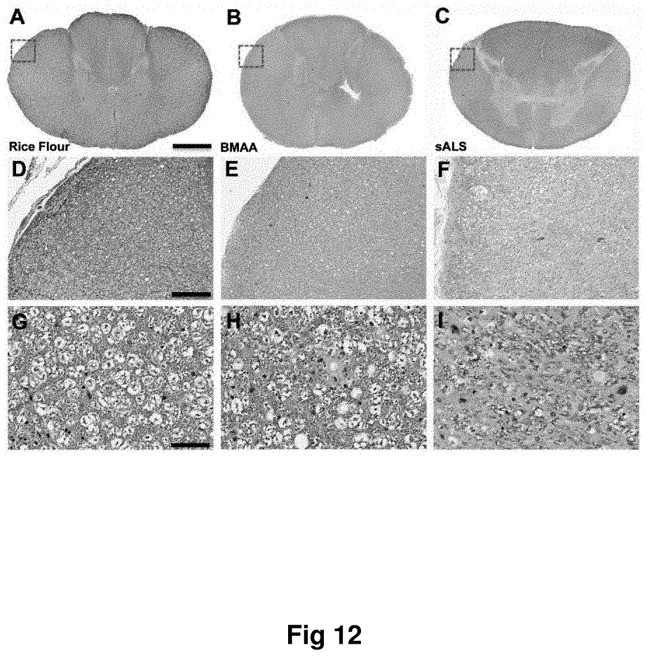

CD68, a marker of proinflammatory microglial activation, was bilaterally expressed in the lateral corticospinal tracts in both cervical and lumbar segments of the spinal cord of BMAA-dosed vervets (FIG. 10; G-I). CD68⁺ microglial density and distribution observed in BMAA-dosed vervets were similar to those seen in a representative autopsy specimen from an individual with neuropathologically confirmed sALS (FIG. 10; J-L). CD68⁺ nodules had increased number and size (p≤0.0001) compared to those of control vervets (FIG. 10; G-I and FIG. 11; G). In addition to proinflammatory microglial activation, we observed pallor of myelin staining in the pyramidal tracts suggesting mild loss of myelinated axon fibers in the lateral corticospinal tracts in 7 of 16 (44%) of vervets fed BMAA (p=0.0956) (FIG. 12 and data not shown). The mean Iba1⁺ microglia distribution and CD68⁺ microglial expression in the lateral corticospinal tract stimulated by BMAA dosing was attenuated 32% and 24%, respectively by coadministration of L-serine in the diet (FIG. 11 and Table 10).

### Corticospinal Pathology

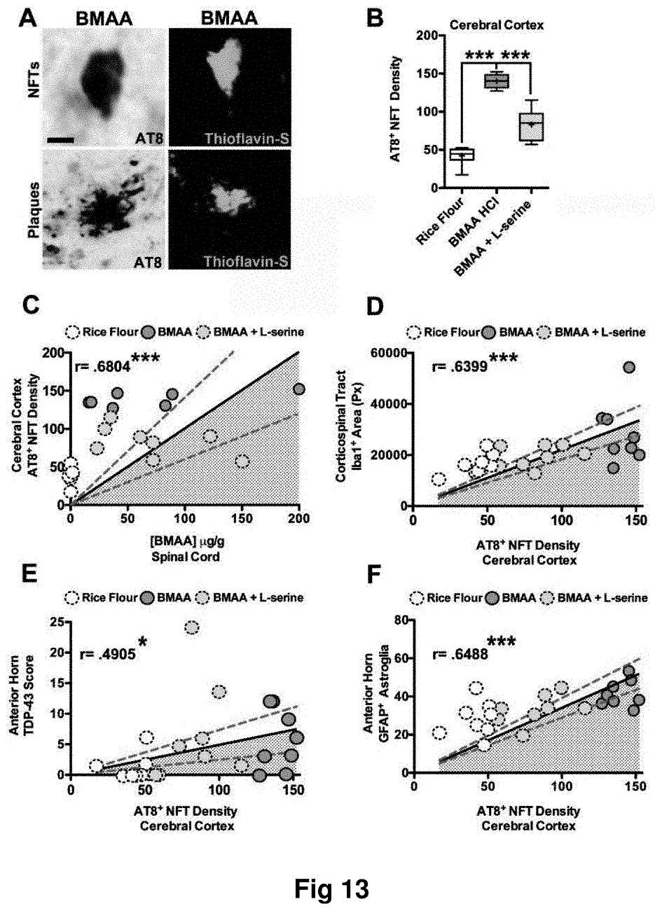

Guam ALS/PDC is characterized by the presence of dense and widely distributed cortical NFTs affecting motor, sensory, and association areas of the cerebral cortex. Chronic dietary exposure to BMAA triggered cortical NFTs in the vervet primate with a density and distribution similar to Guam ALS/PDC. To investigate the relationship between BMAA-induced cortical and spinal cord pathologies, the median tau AT8⁺ NFT density in the same vervets was calculated across 7 cortical brain regions. Chronic BMAA exposure increased the median tau ATV NFT density by 3.1-fold (p<0.0001) and was attenuated 40% by coadministration of L-serine (FIG. 13; A, B). Cortical ATV NFT density deposition observed in BMAA-dosed vervets was also positively correlated with the concentration of BMAA detected in the spinal cord as a marker of exposures (r=0.6804, p=0.0004) (FIG. 13; C) and the ALS/MND-type pathology observed in descending white matter tracts and the anterior horns. Cortical AT8⁺ NFT density was positively correlated with the total area of IbA1⁺ microglia in the lateral cortical spinal tract (r=0.6399, p=0.0004) (FIG. 13; D), the density and distribution of TDP-43⁺ cytoplasmic inclusion in motor neurons (r=0.4905, p=0.01) (FIG. 13; E), and the number of reactive GFAP⁺ astroglia adjacent to those motor neurons (r=0.6488, p=0.0003) (FIG. 13; F). In all these correlation analyses, coadministration of L-serine decreased the degree of the ALS/MND-type pathological changes observed in the cerebral cortex, anterior horn, and lateral corticospinal spinal tracts (FIG. 13; C-F). These observations suggest that chronic dietary exposure to BMAA induces degeneration of upper and lower motor neurons in vervets and coadministration of L-serine reduces pathological changes.

Our results show that chronic dietary exposure to the cyano-toxin BMAA induces ALS/MND-type degeneration of the upper and lower motor neurons in the vervet model. This includes protein inclusions in anterior horn motor neurons, lower motor neuron degeneration, reactive astrogliosis, and microglial activation characteristic of axonal damage and loss of myelinated fibers in the primary descending motor pathways of the vervet spinal cord. The vervet BMAA toxin exposure model recapitulates the features of Guam ALS/PDC and ALS/MND neuropathology including degeneration of both upper and lower motor neurons and TDP-43⁺ neuronal inclusions. It is noteworthy to mention that the vervet primate carries the APOE4 genotype, which may explain the prevalence of cortical AT8⁺ NFTs, in keeping with the cortical dementia associated with Guam ALS/PDC. These data suggest that BMAA-exposed vervets serve as a useful experimental model for testing novel therapeutics for the treatment of ALS/MND.

In the vervet primate, L-serine attenuates the occurrence of NFTs in upper motor neurons of the primary motor cortex. In the spinal cord, we observed rare NFTs in BMAA-fed vervets, consistent with what has been observed in Guamanian ALS/PDC patients. In addition, we found evidence of other neuronal protein inclusions characteristic of ALS/MND, including TDP-43⁺, FUS⁺, UBIQ⁺, and Bunina bodies.

TABLE 9
Quantitative Analysis of pTDP-43⁺ Anterior Horn Motor Neurons

| | Cervical | Lumbar | All Spinal |
|---|---|---|---|
| Exposure Cohort | Segment | Segment | Cord Segments |
| Rice flour | 0.0 ± 0.8 | 0.0 ± 0.8 | 0.0 ± 0.7 |
| BMAA | 3.0 ± 1.9 | 3.0 ± 3.1 | 4.5 ± 1.7* |
| BMAA + L-serine | 3.0 ± 2.8 | 4.5 ± 3.6 | 3.8 ± 1.1^{NS} |

Median ± SEM of the number of TDP-43⁺ motor neurons per region of interest. n 1/4 8 (rice flour vs BMAA);
^{NS}no statistically significant differences were found using the Mann-Whitney U test.
*p = 0.02

L-Serine coadministration with BMAA reduced the number of anterior horn neuron protein inclusions, microglial activation, and reactive astrogliosis. Moreover, L-serine protected against the overall development of cortical NFTs and reduced pathology leading to axonal damage in the lateral corticospinal tracts in the vervet BMAA model.

In vitro, L-serine inhibits misincorporation of BMAA into proteins and modulates the endoplasmic reticulum unfolding protein response. In our vervet study, coadministration of L-serine did not reduce the amount of free BMAA nor did it decrease the detection of BMAA measured in the protein fraction. This observation suggests that alternative mechanisms of neuroprotection, unrelated to protein mis-folding or dysfunction may explain the results observed in vervet primates. Other possible mechanisms for L-serine neuro protection include proliferation of oligodendrocytes stimulating myelin repair or restoring serine homeostasis through racemase conversion of L- to D-serine. L-Serine uptake by astroglia may lead to a compensatory increase in D-serine following dietary BMAA exposures. D-Serine is a glutamate antagonist at AMPA/kainate receptors, which could offset the excitotoxic effects of BMAA binding to NMDA receptors on motor neurons.

Our results provide preclinical evidence of the efficacy of L-serine for treating ALS/MND and show that BMAA exposure to vervet primates provides a useful model for testing compounds to slow or reverse the progression of motor neuron damage.

In conclusion, we demonstrate that chronic dietary exposure to the cyanobacterial toxin BMAA causes degeneration of the upper and lower motor neurons, activates microglia in the lateral corticospinal tracts, and induces proteinopathies with reactive astrogliosis in the anterior horns of the spinal cord that are characteristic of ALS/MND. The BMAA-dosed vervet can be used to model the neuropathology of ALS/MND. The reduction of BMAA triggered pathology by L-serine supports the use of this essential amino acid as a therapeutic intervention to slow progression of the early stages of ALS/MND.

TABLE 10
Quantitative Analysis of IbA1+ Microglia in the Lateral Corticospinal
Tracts

| | Total Area (Px) | IbA1+ Density (Ct) | Size (Px) |
|---|---|---|---|
| Cervical Segment | | | |
| Rice flour | 16 067.5 ± 1442.3 | 1084.3 ± 138.8 | 11.9 ± 1.1 |
| BMAA | 24 839.4 ± 4357.2* | 1831.8 ± 206.9* | 15.4 ± 1.7^{NS} |
| BMAA + L-serine | 19 719.4 ± 1464.9* | 1691.1 ± 137.3^{NS} | 14.1 ± 1.1 |
| Lumbar Segment | | | |
| Rice flour | 15 285.4 ± 1336.9 | 1398.3 ± 257.3 | 14.6 ± 7.5 |
| BMAA | 17 092.1 ± 2194.3^{NS} | 1213.7 ± 151.5^{NS} | 13.9 ± 1.9^{NS} |
| BMAA + L-serine | 12 379.3 ± 1388.1 | 963.9 ± 152.2 | 11.8 ± 4.8 |

Median ± SEM of IbA1⁺ microglia automated measurements.
n = 8 vervets per group;
ANOVA test (cervical segments);
^{NS}no statistical significance using Kruskal-Wallis test (lumbar segments);
Ct, counts;
Px, pixels.
*p ≤ 0.05.

### Example 5—Significant Changes in miRNA Expression are not Observed in PD or AD

Neural enriched extracellular vesicles from the blood of plasma of six Parkinson's Disease (PD) patients and six control samples from persons who died without neurological disease were analyzed using the methods described in Example 3. The disease status was confirmed by neuropathology. All blood samples came from autopsy patient blood. RNA cargo was extracted from the extracellular vesicles, the indicated eight miRNA sequences were analyzed for expression and the fold expression 2(power-ddCt) was compared between controls and PD patients. The eight miRNA sequences were the same eight miRNA identified in Example 3 that differentiate amyotrophic lateral sclerosis (ALS) patients from controls (i.e., miRNA: miR-10b-5p, miR-146a-5p, miR-199a-3p, miR-4454, miR-29b-3p, miR-151 a-3p, miR-151a-5p, and miR-199a-5p).

The data in Table 11 below indicates that the expression of the eight indicated miRNAs were not significantly different between patients with Parkinson's Disease and control samples using a Mann-Whitney U test.

TABLE 11
Parkinson's Disease

| | | median | | | |
|---|---|---|---|---|---|
| | median PD- | control- | | | |
| | autopsy fold | autopsy fold | | | |
| | expression | expression | | | |
| | 2(power- | 2(power- | | | Significant |
| miRNA | ddCt) | ddCt) | U-value | Z-score | at p < 0.05 |
| 10b-5p | 0.26 | 1.27 | 3 | 1.88 | No |
| 146a-5p | 0.22 | 1.01 | 4 | 1.67 | No |
| 199a-3p | 0.73 | 1.19 | 6 | 1.25 | No |
| 4454 | 2.27 | 1.03 | 9 | −0.62 | No |
| 29-3p | 1.47 | 1.07 | 7 | −1.04 | No |
| 151a-3p | 1.92 | 2.22 | 17 | 0.00 | No |
| 151a-5p | 0.69 | 1.14 | 9 | 0.63 | No |
| 199a-5p | 0.86 | 1.24 | 8 | 0.83 | No |

Neural enriched extracellular vesicles from the blood of plasma of ten Alzheimer's patients (at their initial visit to a neurologist) and ten healthy controls were analyzed using the methods described in Example 3. RNA cargo was extracted from the extracellular vesicles and expression of the eight indicated miRNA sequences was analyzed. Fold expression 2(power-ddCt) was compared between controls and Alzheimer's patients (AD). The data of Table 12 shows that the expression of the eight indicated miRNAs did not significantly differ between AD patients and controls using a Mann-Whitney U test.

TABLE 12
Alzheimer's Disease

| | median | median | | | |
|---|---|---|---|---|---|
| | AD fold | control fold | | | |
| | expression | expression | | | |
| | 2(power- | 2(power- | | | Significant |
| stats | ddCt) | ddCt) | U-value | Z-score | at p < 0.05 |
| 10b-5p | 1.59 | 1.35 | 39 | −0.79 | No |
| 146a-5p | 1.54 | 0.81 | 37 | −0.94 | No |
| 199a-3p | 1.28 | 1,28 | 45 | 0.34 | No |
| 4454 | 1.21 | 1.36 | 49 | 0.04 | No |
| 29-3p | 0.98 | 0,98 | 49 | −0.04 | No |
| 151a-3p | 3.53 | 1.31 | 31 | −1.40 | No |
| 151a-5p | 1.83 | 1.28 | 25 | −1.85 | No |
| 199a-5p | 2.45 | 0.99 | 30 | −1.47 | No |

This data demonstrates that the of changes of expression observed for miR-10b-5p, miR-146a-5p, miR-199a-3p, miR-4454, miR-29b-3p, miR-151a-3p, miR-151a-5p, and miR-199a-5p in the ALS patients of Example 3, are specific to ALS patients, and such changes are not observed in AD or PD patients.

### Example 6—Additional Embodiments

A1. A method of detecting one or more nucleic acids in a human subject comprising:
(a) preparing a neural-enriched exosome fraction from a sample obtained from the subject;
(b) detecting and/or quantitating a presence or amount of the one or more nucleic acids associated with the neural-enriched exosome fraction, wherein the one or more nucleic acids comprise a micro-RNA selected from the group consisting of miR-146a-5p, miR-199a-3p, miR-4454, miR-10b-5p, miR-29b-3p, miR-151a-3p, miR-151a-5p, and miR-199a-5p, and
(c) determining the presence or absence of a neurodegenerative disease in the subject, according to the presence or amount of the one or more nucleic acids detected, wherein when the presence of a neurodegenerative disease in the subject is determined in (b), the method comprises (d) treating the neurodegenerative disease by a process comprising administering a therapeutically effective amount of a drug selected from one or more of ralitoline, phenytoin, lamotrigine, carbamazepine, lidocaine, tetrodotoxin, nitroindazole, a sulforaphane or sulforaphane analogue, gabapentin, pregabalin, Mirogabalin, gabapentin enacarbil, phenibut, imagabalin, atagabalin, 4-methylpregabalin, PD-217,014, riluzole, edaravone, tetrabenazine, haloperidol, risperidone, quetiapine, amantadine, levetiracetam, clonazepam, citalopram, escitalopram, fluoxetine, sertraline, quetiapine, risperidone, olanzapine, valproate, carbamazepine, lamotrigine, a vaccine, a cholinesterase inhibitor, memantine, an antidepressant, an N-methyl D-aspartate (NMDA) antagonist, an omega-3 fatty acid, curcumin, or a curcumin derivative, vitamin E, a sleep aid, an anti-anxiety drug, an anti-convulsant, an anti-psychotic, carbidopa-levodopa, amantadine, a dopamine agonists, a MAO B inhibitor, a Catechol O-methyltransferase (COMT) inhibitor, and an anticholinerigic.
B2. A method of determining whether a subject has, or is at risk of developing, a neurodegenerative disorder, the method comprising:
(a) preparing a neural-enriched exosome fraction from a sample obtained from the subject;
(b) detecting and/or quantitating a presence or amount of the one or more nucleic acids associated with the neural-enriched exosome fraction; and
(c) measuring the presence or amount of the one or more of the nucleic acids;
wherein a result of performing step (c) that is about 20-fold, about 19-fold, about 18-fold, about 17-fold, about 16-fold, about 15-fold, about 14-fold, about 13 fold, about 12 fold, about 11 fold, about 10.5 fold, about 10 fold, about 9.5 fold, about 9 fold, about 8.5 fold, about 8 fold, about 7.5 fold, about 7 fold, about 6.5 fold, about 6 fold, about 5.5 fold, about 5 fold, about 4.5 fold, about 4 fold, about 3.5 fold, about 3 fold, about 2.9 fold, about 2.8 fold, about 2.7 fold, about 2.6 fold, about 2.5 fold, about 2.4 fold, about 2.3 fold, about 2.2 fold, about 2 fold, about 1.9 fold, about 1.8 fold, about 1.7 fold, about 1.6 fold, about 1.5 fold, about 1.4 fold, about 1.3 fold, about 1.2, or about 1.1 fold higher or lower than a baseline amount of such one or more nucleic of baseline indicates that the subject has, or is a risk of developing, the neurodegenerative disorder.
C3. The method of embodiment B2, wherein the baseline is determined by, comparing the amount of the one or more nucleic acids detected in (b) to an amount of the one or more nucleic acids obtained from at least one control subject.
D4. The method of embodiment B2 or embodiment C3, wherein the method further comprises
(d) treating the neurodegenerative disease by a process comprising administering a therapeutically effective amount of a drug selected from one or more of ralitoline, phenytoin, lamotrigine, carbamazepine, lidocaine, tetrodotoxin, nitroindazole, a sulforaphane or sulforaphane analogue, gabapentin, pregabalin, Mirogabalin, gabapentin enacarbil, phenibut, imagabalin, atagabalin, 4-methylpregabalin, PD-217,014, riluzole, edaravone, tetrabenazine, haloperidol, risperidone, quetiapine, amantadine, levetiracetam, clonazepam, citalopram, escitalopram, fluoxetine, sertraline, quetiapine, risperidone, olanzapine, valproate, carbamazepine, lamotrigine, a vaccine, a cholinesterase inhibitor, memantine, an antidepressant, an N-methyl D-aspartate (NMDA) antagonist, an omega-3 fatty acid, curcumin, or a curcumin derivative, vitamin E, a sleep aid, an anti-anxiety drug, an anti-convulsant, an anti-psychotic, carbidopa-levodopa, amantadine, a dopamine agonists, a MAO B inhibitor, a Catechol O-methyltransferase (COMT) inhibitor, and an anticholinerigic.
E5. The method of any one of embodiments B2-D4, wherein the one or more nucleic acids comprise a micro-RNA selected from the group consisting of miR-146a-5p, miR-199a-3p, miR-4454, miR-10b-5p, miR-29b-3p, miR-151a-3p, miR-151a-5p, and miR-199a-5p.
F6. The method of any one of embodiments A1-E5, wherein the neurodegenerative disorder is ALS.
G7. A method of detecting one or more nucleic acids in a subject comprising:

(a) preparing a neural-enriched exosome fraction from a sample obtained from the subject; and

(b) detecting and/or quantitating a presence or amount of the one or more nucleic acids associated with the neural-enriched exosome fraction.
G8. The method of embodiment G7, wherein the one or more nucleic acids comprise one or more micro-RNAs selected from the group consisting of miR-146a-5p, miR-199a-3p, miR-4454, miR-10b-5p, miR-29b-3p, miR-151a-3p, miR-151a-5p, and miR-199a-5p.
G9. The method of embodiment G8, wherein the one or more nucleic acids comprise one, two, three, four, five, six, seven or eight micro-RNAs selected from the group consisting of miR-146a-5p, miR-199a-3p, miR-4454, miR-10b-5p, miR-29b-3p, miR-151a-3p, miR-151a-5p, and miR-199a-5p.
H8. A method of detecting one or more nucleic acids in a subject comprising:

(a) detecting a neural-enriched exosome fraction in a sample obtained from the subject; and

(b) detecting and/or quantitating a presence or amount of the one or more nucleic acids associated with the neural-enriched exosome fraction.
I9. A method of detecting one or more nucleic acids in a subject comprising:

(a) isolating a neural-enriched exosome fraction prepared from a sample obtained from the subject; and

(b) detecting and/or quantitating a presence or amount of the one or more nucleic acids associated with the neural-enriched exosome fraction.
J10. The method of any one of embodiments G7 to 19, further comprising (c), comparing the amount of the one or more nucleic acids detected in (b) to a baseline amount of the one or more nucleic acids associated with the neural-enriched exosome fraction.
K11. The method of any one of embodiments G7 to J10, wherein the one or more nucleic acids comprises messenger RNA (mRNA), microRNA (miRNA), and/or small interfering RNA (siRNA).
L12. The method of any one of embodiments G7 to K11, wherein the one or more nucleic acids comprises one or more miRNAs selected from the group consisting of miR-146a-5p, miR-199a-3p, miR-4454, miR-10b-5p, miR-29b-3p, miR-151a-3p, miR-151a-5p, and miR-199a-5p.
L13. The method of embodiment L12, wherein the one or more nucleic acids comprise one, two, three, four, five, six, seven or eight micro-RNAs selected from the group consisting of miR-146a-5p, miR-199a-3p, miR-4454, miR-10b-5p, miR-29b-3p, miR-151a-3p, miR-151a-5p, and miR-199a-5p.
M13. The method of any one of embodiments A1 to L12, wherein the subject is a mammal.
N14. The method of any one of embodiments A1 to M13, wherein the subject is a human.
O15. The method of any one of embodiments A1 to N14, wherein the method is an ex vivo method.
P16. The method of any one of embodiments A1 to O15, wherein the method is an in vitro method.
Q17. The method of any one of embodiments A1 to P16 wherein the neural-enriched exosome fraction if prepared from a bodily fluid obtained from the subject.
R18. The method of embodiment Q17, wherein the bodily fluid comprises or consists of blood, or a blood product.
S19. The method of embodiment R18, wherein the blood product is plasma.
T20. The method of embodiments Q17, wherein the bodily fluid is cerebral spinal fluid.
U21. The method of any one of embodiments A1 to T20, wherein the sample comprises brain or neural tissue.
V22. The method of any one of embodiments A1 to U21, wherein the neural-enriched exosome fraction is prepared (a) by a process comprising immunoprecipitating the neural-enriched exosome fraction using a binding agent that specifically binds to a tetraspanin or a cell adhesion molecule.
W23. The method of embodiment V22, wherein the tetraspanin is selected from the group consisting of CD9, CD63, and CD81.
X24. The method of embodiment V22, wherein the cell adhesion molecule is selected from the group consisting of a cadherin, a nectin, a sidekick cell adhesion molecule, an integrin, a neuroligin, a neuroexin, an ephrin, Syg-1, Syg-2, L1CAM/CD171, and NCAM/CD56.
Y25. The method of embodiment V22, wherein the cell adhesion molecule is L1CAM/CD171.
Z26. The method of embodiment V22 or embodiment Y25, wherein the cell adhesion molecule is NCAM/CD56.
AA27. The method of embodiment B2-F6 and J10-Z26, wherein the baseline amount comprises an amount of the one or more nucleic acids associated with a neural-enriched exosome fraction obtained from at least one control subject.
BB28. The method of embodiment any one of embodiments G7 to V22, further comprising determining the absence of a neurodegenerative disease in the subject according to the amount of the one or more nucleic acids detected in the neural-enriched exosome fraction.
CC29. The method of embodiment J10, further comprising (d) determining: the presence of; the absence of; or risk of developing; a neurodegenerative disease in the subject according to the comparison of (c).
DD30. The method of embodiment J10, wherein the comparison of (c) shows a higher amount of the one or more nucleic acids in the subject compared to the control subject.
EE31. The method of embodiment DD30, wherein the amount is about 20-fold, about 19-fold, about 18-fold, about 17-fold, about 16-fold, about 15-fold, about 14-fold, about 13 fold, about 12 fold, about 11 fold, about 10.5 fold, about 10 fold, about 9.5 fold, about 9 fold, about 8.5 fold, about 8 fold, about 7.5 fold, about 7 fold, about 6.5 fold, about 6 fold, about 5.5 fold, about 5 fold, about 4.5 fold, about 4 fold, about 3.5 fold, about 3 fold, about 2.9 fold, about 2.8 fold, about 2.7 fold, about 2.6 fold, about 2.5 fold, about 2.4 fold, about 2.3 fold, about 2.2 fold, about 2 fold, about 1.9 fold, about 1.8 fold, about 1.7 fold, about 1.6 fold, about 1.5 fold, about 1.4 fold, about 1.3 fold, about 1.2, or about 1.1 fold higher than the amount of the one or more nucleic acids in the subject compared to the control subject.
FF32. The method of embodiment EE31, wherein the subject is determined to have or to be at risk of developing the neurodegenerative disease.
GG33. The method embodiment FF32, where the neurodegenerative disease is selected from Alzheimer's disease (AD), Parkinson's disease, Amyotrophic Lateral Sclerosis (ALS), Huntington's disease (HD), multiple sclerosis, myotropic lateral sclerosis, amyotrophic lateral sclerosis, Pick's disease, spinocerebellar atrophy, Machado-Joseph's disease, denatorubropallidoluysian atrophy, Creutzfeldt-Jakob's disease, and Lewy body disease.
HH34. The method of embodiment GG33, wherein the neurodegenative disease is selected from Alzheimer's disease (AD), Parkinson's disease, Amyotrophic Lateral Sclerosis (ALS) and Huntington's disease (HD).
HH35. The method of embodiment GG33, wherein the neurodegenative disease is Amyotrophic Lateral Sclerosis (ALS).
HH36. The method of embodiment GG33, wherein the neurodegenative disease is not Alzheimer's disease (AD) or Parkinson's disease.
HH37. The method of embodiment GG33, wherein the neurodegenative disease is not a disease selected from one or more of Huntington's disease (HD), multiple sclerosis, myotropic lateral sclerosis, amyotrophic lateral sclerosis, Pick's disease, spinocerebellar atrophy, Machado-Joseph's disease, denatorubropallidoluysian atrophy, Creutzfeldt-Jakob's disease, and Lewy body disease.
II35. The method of embodiment J10, wherein the baseline is determined by comparing the amount of the one or more nucleic acids detected in (b) to an amount of the one or more nucleic acids associated with a neural-enriched exosome fraction obtained from at least one control subject.
JJ36. The method of embodiment CC29, wherein when the presence of the neurodegenerative disease is determined and/or wherein the subject is determined to have or to be at risk of developing the neurodegenerative disease, the method further comprises treating the neurodegenerative disease or one or more symptoms thereof.
KK37. The method of embodiment JJ36, wherein the treatment comprises administering a therapeutically effective amount of a drug selected from one or more of ralitoline, phenytoin, lamotrigine, carbamazepine, lidocaine, tetrodotoxin, nitroindazole, a sulforaphane or sulforaphane analogue, gabapentin, pregabalin, Mirogabalin, gabapentin enacarbil, phenibut, imagabalin, atagabalin, 4-methylpregabalin, PD-217,014, riluzole, edaravone, tetrabenazine, haloperidol, risperidone, quetiapine, amantadine, levetiracetam, clonazepam, citalopram, escitalopram, fluoxetine, sertraline, quetiapine, risperidone, olanzapine, valproate, carbamazepine, lamotrigine, a vaccine, a cholinesterase inhibitor, memantine, an antidepressant, an N-methyl D-aspartate (NMDA) antagonist, an omega-3 fatty acid, curcumin, or a curcumin derivative, vitamin E, a sleep aid, an anti-anxiety drug, an anti-convulsant, an anti-psychotic, carbidopa-levodopa, amantadine, a dopamine agonists, a MAO B inhibitor, a Catechol O-methyltransferase (COMT) inhibitor, and an anticholinerigic.
LL38. The method of embodiment KK37, wherein the neurodegenerative disease is ALS and the treatment comprises administering a therapeutically effective amount of L-serine, ralitoline, phenytoin, lamotrigine, carbamazepine, lidocaine, tetrodotoxin, Riluzole, Edaravone, Gabapentin, pregabalin, Mirogabalin, gabapentin enacarbil, phenibut, imagabalin, atagabalin, 4-methylpregabalin, PD-217,014, Trihexyphenidyl, amitriptyline, baclofen, diazepam or CK-2127107.
MM39. The method of embodiment KK37, wherein the neurodegenerative disease is HD and the treatment comprises administered a therapeutically effective amount of tetrabenazine, haloperidol, risperidone, quetiapine, amantadine, levetiracetam, clonazepam, citalopram escitalopram, fluoxetine, sertraline, quetiapine, risperidone, olanzapine, valproate, carbamazepine, or lamotrigine.
NN40. The method of embodiment KK37, wherein the neurodegenerative disease is AD and the treatment comprises administered a therapeutically effective amount of a vaccine, a cholinesterase inhibitor, memantine, an antidepressant, an N-methyl D-aspartate (NMDA) antagonist, omega-3 fatty acids, curcumin, or a curcumin derivative, vitamin E, a sleep aid, an anti-anxiety drug, an anti-convulsant, or an anti-psychotic.
OO41. The method of embodiment KK37, wherein the neurodegenerative disease is PD and the treatment comprises administering a therapeutically effective amount of carbidopa-levodopa, amantadine, a dopamine agonists, a MAO B inhibitor, a Catechol O-methyltransferase (COMT) inhibitor or an anticholinergic.
PP42. A method comprising detecting and/or quantitating a presence, absence or amount of one or more miRNAs selected from the group consisting of miR-146a-5p, miR-199a-3p, miR-4454, miR-10b-5p, miR-29b-3p, miR-151a-3p, miR-151a-5p, and miR-199a-5p associated with a neural-enriched exosome fraction obtained from a subject, wherein the presence or amount of the one or more miRNAs indicates that the subject has ALS.

The entirety of each patent, patent application, publication or any other reference or document cited herein hereby is incorporated by reference. In case of conflict, the specification, including definitions, will control.

Citation of any patent, patent application, publication or any other document is not an admission that any of the foregoing is pertinent prior art, nor does it constitute any admission as to the contents or date of these publications or documents.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

All of the features disclosed herein may be combined in any combination. Each feature disclosed in the specification may be replaced by an alternative feature serving a same, equivalent, or similar purpose. Thus, unless expressly stated otherwise, disclosed features (e.g., antibodies) are an example of a genus of equivalent or similar features.

As used herein, all numerical values or numerical ranges include integers within such ranges and fractions of the values or the integers within ranges unless the context clearly indicates otherwise. Further, when a listing of values is described herein (e.g., about 50%, 60%, 70%, 80%, 85% or 86%) the listing includes all intermediate and fractional values thereof (e.g., 54%, 85.4%). Thus, to illustrate, reference to 80% or more, includes 81%, 82%, 83%, 84%, 85%, 86%, 87%, 88%, 89%, 90%, 91%, 92%, 93%, 94% etc., as well as 81.1%, 81.2%, 81.3%, 81.4%, 81.5%, etc., 82.1%, 82.2%, 82.3%, 82.4%, 82.5%, etc., and so forth.

Reference to an integer with more (greater) or less than includes any number greater or less than the reference number, respectively. Thus, for example, a reference to less than 100, includes 99, 98, 97, etc. all the way down to the number one (1); and less than 10, includes 9, 8, 7, etc. all the way down to the number one (1).

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth.

Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-1,000, 1,000-1,500, 1,500-2,000, 2,000-2,500, 2,500-3,000, 3,000-3,500, 3,500-4,000, 4,000-4,500, 4,500-5,000, 5,500-6,000, 6,000-7,000, 7,000-8,000, or 8,000-9,000, includes ranges of 10-50, 50-100, 100-1,000, 1,000-3,000, 2,000-4,000, etc.

Modifications can be made to the foregoing without departing from the basic aspects of the technology. Although the technology has been described in substantial detail with reference to one or more specific embodiments, those of ordinary skill in the art will recognize that changes can be made to the embodiments specifically disclosed in this application, yet these modifications and improvements are within the scope and spirit of the technology.

The invention is generally disclosed herein using affirmative language to describe the numerous embodiments and aspects. The invention also specifically includes embodiments in which particular subject matter is excluded, in full or in part, such as substances or materials, method steps and conditions, protocols, or procedures. For example, in certain embodiments or aspects of the invention, materials and/or method steps are excluded. Thus, even though the invention is generally not expressed herein in terms of what the invention does not include aspects that are not expressly excluded in the invention are nevertheless disclosed herein.

The technology illustratively described herein suitably can be practiced in the absence of any element(s) not specifically disclosed herein. Thus, for example, in each instance herein any of the terms “comprising,” “consisting essentially of,” and “consisting of” can be replaced with either of the other two terms. Some embodiments of the technology described herein suitably can be practiced in the absence of an element not specifically disclosed herein. Accordingly, in some embodiments the term “comprising” or “comprises” can be replaced with “consisting essentially of” or “consisting of” or grammatical variations thereof. A composition “consisting essentially of” refers to a composition that includes only the active ingredients claimed (e.g., active ingredient (AI) or active pharmaceutical ingredient (API); e.g., L-serine, a salt, metabolic precursor, derivative or conjugate thereof); which composition may include other ingredients such as formulation materials, excipients, additives, carriers, preservatives, diluents, solvents, fillers, salts, buffers, coatings, binders, and lubricating agents; and which composition excludes other APIs not claimed.

The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%), and use of the term “about” at the beginning of a string of values modifies each of the values (i.e., “about 1, 2 and 3” refers to about 1, about 2 and about 3). For example, a weight of “about 100 grams” can include weights between 90 grams and 110 grams. The term, “substantially” as used herein refers to a value modifier meaning “at least 95%”, “at least 96%”, “at least 97%”, “at least 98%”, or “at least 99%” and may include 100%. For example, a composition that is substantially free of X, may include less than 5%, less than 4%, less than 3%, less than 2%, or less than 1% of X, and/or X may be absent or undetectable in the composition.

Thus, it should be understood that although the present technology has been specifically disclosed by representative embodiments and optional features, modification and variation of the concepts herein disclosed can be resorted to by those skilled in the art, and such modifications and variations are considered within the scope of this technology.

What is claimed is:

1. A method of identifying a subject who has, or is at risk of developing a motor neuron disease comprising:
(a) determining a presence or amount of one or more micro-RNAs (miRNAs) in a sample obtained from the subject wherein the one or more miRNA are selected from the group consisting of miR-146a-5p, miR-199a-3p, miR-4454, miR-10b-5p, miR-29b-3p, miR-151a-3p, miR-151a-5p, and miR-199a-5p, and
(b) determining if the subject has, or is at risk of developing the motor neuron disease according to the presence or amount of the one or more miRNAs in the sample.

2. The method of claim 1, wherein the method further comprises A method of preventing or treating a motor neuron disease in a subject who has, or is at risk of developing the motor neuron disease, the method comprising:
(c) administering therapeutically effective amount of a motor neuron disease drug to the subject when the determining of (b) determines that the subject has, or is at risk of developing the motor neuron disease.

3. The method of claim 2, wherein the motor neuron disease drug is selected from L-serine, ralitoline, phenytoin, lamotrigine, carbamazepine, lidocaine, tetrodotoxin, nitroindazole, a sulforaphane or sulforaphane analogue, gabapentin, pregabalin, Mirogabalin, gabapentin enacarbil, phenibut, imagabalin, atagabalin, 4-methylpregabalin, PD-217,014, riluzole, edaravone, tetrabenazine, haloperidol, risperidone, quetiapine, amantadine, levetiracetam, clonazepam, citalopram, escitalopram, fluoxetine, sertraline, quetiapine, risperidone, olanzapine, valproate, carbamazepine, lamotrigine, a vaccine, a cholinesterase inhibitor, memantine, an antidepressant, an N-methyl D-aspartate (NMDA) antagonist, an omega-3 fatty acid, curcumin, or a curcumin derivative, vitamin E, a sleep aid, an anti-anxiety drug, an anti-convulsant, an anti-psychotic, carbidopa-levodopa, amantadine, a dopamine agonists, a MAO B inhibitor, a Catechol O-methyltransferase (COMT) inhibitor, and an anticholinerigic.

4. The method of claim 1, wherein the sample comprises neural-derived exosomes.

5. The method of any one of claim 1, wherein the miRNAs are obtained from, or derived from, neural-derived exosomes.

6. The method of claim 1, further comprising, prior to (a), preparing neural-derived exosomes from the sample obtained from the subject.

7. The method of claim 6, further comprising isolating miRNA from the neural-derived exosomes.

8. The method of claim 1, wherein the amount of the one or more micro-RNAs determined in (a) is at least 1.5-fold higher or lower than a baseline amount thereby indicating the subject has, or is a risk of developing, the motor neuron disease.

9. The method of claim 1, wherein the motor neuron disease is ALS.

10. The method of claim 1, wherein the subject is a human.

11. The method of a claim 1, wherein the subject is asymptomatic for the motor neuron disease.

12. The method of claim 1, wherein the baseline amount is an average, mean or absolute amount of the one or more miRNAs present in a healthy control subject.

13. The method of claim 1, wherein the determining of (a) determines a presence or amount of three or more of the miRNAs in the sample.

14. The method of claim 1, wherein the neural-derived exosomes comprise a tetraspanin.

15. The method of claim 1, wherein the tetraspanin is selected from the group consisting of CD9, CD63, and CD81.

16. The method of claim 1, wherein the neural-derived exosomes comprise a cell adhesion molecule selected from the group consisting of a cadherin, a nectin, a sidekick cell adhesion molecule, an integrin, a neuroligin, a neuroexin, an ephrin, Syg-1, Syg-2, L1CAM/CD171, and NCAM/CD56.

17. The method of claim 1, further comprising determining the absence of the motor neuron disease in the subject according to the presence or amount of the one or more miRNAs in the sample.

18. The method of claim 1, further comprising monitoring the progression of the motor neuron disease in the subject, wherein the method is conducted two or more times for the subject.

19. The method of claim 1, wherein the subject is not diagnosed with a motor neuron disease prior to the determining of (a) or (b).

20. The method of claim 2, wherein the treating of the motor neuron disease comprises inhibiting or delaying the onset or progression of the motor neuron disease.

21. The method of claim 2, wherein the motor neuron disease is selected from one or more of Alzheimer's disease (AD), Parkinson's disease, Amyotrophic Lateral Sclerosis (ALS), ALS/PCD, Huntington's disease (HD), multiple sclerosis, myotropic lateral sclerosis, amyotrophic lateral sclerosis, Pick's disease, spinocerebellar atrophy, Machado-Joseph's disease, denatorubropallidoluysian atrophy, Creutzfeldt-Jakob's disease, and Lewy body disease.

22. A method of preventing or treating a motor neuron disease in a subject who has, or is at risk of developing the motor neuron disease, the method comprising:
(a) determining a presence or amount of one or more micro-RNAs (miRNAs) in a sample obtained from the subject wherein the one or more miRNA are selected from the group consisting of miR-146a-5p, miR-199a-3p, miR-4454, miR-10b-5p, miR-29b-3p, miR-151a-3p, miR-151a-5p, and miR-199a-5p;
(b) determining if the subject has, or is at risk of developing the motor neuron disease according to the presence or amount of the one or more miRNAs in the sample; and
(c) administering a therapeutically effective amount of a motor neuron disease drug to the subject when the determining of (b) determines that the subject has, or is at risk of developing the motor neuron disease.


SEQUENCE LISTING (8 sequences)

SEQ ID NO: 1 - DNA, 22 bp, Homo sapiens
tgagaactgaattccatgggtt

SEQ ID NO: 2 - DNA, 22 bp, Homo sapiens
acagtagtctgcacattggtta

SEQ ID NO: 3 - DNA, 20 bp, Homo sapiens
ggatccgagtcacggcacca

SEQ ID NO: 4 - DNA, 23 bp, Homo sapiens
taccctgtagaaccgaatttgtg

SEQ ID NO: 5 - DNA, 23 bp, Homo sapiens
tagcaccatttgaaatcagtgtt

SEQ ID NO: 6 - DNA, 21 bp, Homo sapiens
ctagactgaagctccttgagg

SEQ ID NO: 7 - DNA, 21 bp, Homo sapiens
tcgaggagctcacagtctagt

SEQ ID NO: 8 - DNA, 23 bp, Homo sapiens
cccagtgttcagactacctgttc